(19) United States
(12) Patent Application Publication
(10) Pub. No.: US 2007/0185078 A1
(43) Pub. Date: Aug. 9, 2007

(54) Substituted triazole derivatives as oxytocin antagonists

(76) Inventors: Alan Daniel Brown, Sandwich (GB); Andrew Antony Calabrese, Sandwich (GB); David Ellis, Sandwich (GB); Lesa Watson, Sandwich (GB)

Correspondence Address: PFIZER INC., PATENT DEPARTMENT, MS8260-1611, EASTERN POINT ROAD, GROTON, CT, 06340, US

(21) Appl. No.: 11/570,199

(22) PCT Filed: May 27, 2005
(86) PCT No.: PCT/IB05/01541

§ 371 Date: Dec. 7, 2006

Related U.S. Application Data
(60) Provisional application No. 60/588,170, filed on Jul. 14, 2004.

(30) Foreign Application Priority Data
Jun. 9, 2004 (GB) 0412874.0

(51) Int. Cl. A61K 31/551; A61K 31/554; C07D 487/12; C07D 498/02

(52) U.S. Cl. 514/211.12; 514/220; 540/548; 540/563

(57) ABSTRACT

This invention relates to compounds of formula (I)

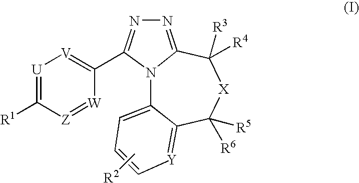

The present invention relates to a class of substituted triazoles with activity as oxytocin antagonists, uses thereof, processes for the preparation thereof and compositions containing said inhibitors. These inhibitors have utility in a variety of therapeutic areas including sexual dysfunction, particularly premature ejaculation (P.E.).

International patent applications PCT/IB2004/002977 and PCT/IB2005/000313 disclose substituted triazoles with activity as oxytocin antagonists.

According to a first aspect, the present invention provides compounds of formula (I) 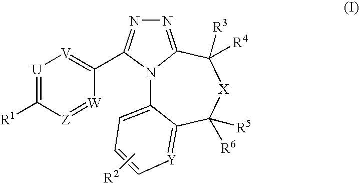
wherein
U, V, W and Z are each independently N or CR⁷;
Y is N or CH;
X is O or NR⁸;
R¹ is selected from
(i) phenyl, which is optionally substituted by one or more groups each independently selected from halo, hydroxy, CN, NO₂, (C₁-C₆)alkyl, (C₁-C₆)alkoxy, (C₁-C₆)haloalkyl, (C₁-C₆)haloalkoxy, COR⁹, CO₂R⁹, NR⁹R¹⁰ and CONR⁹R¹⁰,
(ii) a five to seven membered heteroaromatic ring containing 1 to 3 heteroatoms independently selected from nitrogen, oxygen and sulfur, which is optionally substituted by one or more groups independently selected from halo, hydroxy, CN, NO₂, (C₁-C₆)alkyl, (C₁-C₆)alkoxy, (C₁-C₆)haloalkyl, (C₁-C₆)haloalkoxy, COR⁹, CO₂R⁹, NR⁹R¹⁰ and CONR⁹R¹⁰, and
(iii) (C₁-C₆)alkoxy, which is optionally substituted by one or more substituents each independently selected from (C₁-C₆)alkoxy, halo, hydroxy and phenyl;
R² is hydrogen, halo, hydroxy, CN, NO₂, (C₁-C₆)alkyl, (C₁-C₆)alkoxy, (C₁-C₆)haloalkyl, (C₁-C₆)haloalkoxy, COR⁹, CO₂R⁹, NR⁹R¹⁰ or CONR⁹R¹⁰;
R³, R⁴, R⁵, and R⁶ are each independently hydrogen or (C₁-C₆)alkyl;
R⁷ is independently selected from hydrogen, halo, hydroxy, CN, NO₂, (C₁-C₆)alkyl, (C₁-C₆)alkoxy, (C₁-C₆)haloalkyl, (C₁-C₆)haloalkoxy, COR⁹, CO₂R⁹, NR⁹R¹⁰ or CONR⁹R¹⁰;
R⁸ is hydrogen or (C₁-C₆)alkyl, CO(C₁-C₆)alkyl, CO₂(C₁-C₆)alkyl or SO₂(C₁-C₆)alkyl, each of which is optionally substituted by one or more groups independently selected from halo, hydroxy, (C₁-C₆)alkoxy, CN, NO₂ and phenyl; and
R⁹ and R¹⁰ are each independently hydrogen or (C₁-C₆)alkyl;
   a tautomer thereof or a pharmaceutically acceptable salt, solvate or polymorph of said compound or tautomer or a prodrug thereof.

Unless otherwise indicated, alkyl and alkoxy groups may be straight or branched and contain 1 to 6 carbon atoms and preferably 1 to 4 carbon atoms. Examples of alkyl include methyl, ethyl, n-propyl, isopropyl, n-butyl, isobutyl, sec-butyl, pentyl and hexyl. Examples of alkoxy include methoxy, ethoxy, isopropoxy and n-butoxy. Halo means fluoro, chloro, bromo or iodo and is preferably fluoro. Haloalkyl includes monohaloalkyl, polyhaloalkyl and perhaloalkyl, such as 2-bromoethyl, 2,2,2-trifluoroethyl, chlorodifluoromethyl and trichloromethyl. Haloalkoxy includes monohaloalkoxy, polyhaloalkoxy and perhaloalkoxy, such as 2-bromoethoxy, 2,2,2-trifluoroethoxy, chlorodifluoromethoxy and trichloromethoxy.

Preferably, 1 or 2 of the groups U, V, W and Z represent N when the remainder represent CR⁷.

More preferably, V and Z are CH and U and W are each independently CH or N.

Most preferably, U is CH and W is CH or N.

In a preferred embodiment, U, V, W and Z are CH.

In another preferred embodiment, U, V and Z are CH and W is N.

In a preferred embodiment, Y is N.

In another preferred embodiment, Y is CH.

Preferably, R¹ is selected from
(i) phenyl, which is optionally substituted by one or more groups each independently selected from halo, (C₁-C₆)alkyl, (C₁-C₆)alkoxy, and cyano,
(ii) a six-membered heteroaromatic ring containing 1 to 2 nitrogen atoms, which is optionally substituted by one or more groups each independently selected from halo, (C₁-C₆)alkyl, (C₁-C₆)alkoxy, and cyano; and
(iii) (C₁-C₆)alkoxy, which is optionally substituted by one or more substituents each independently selected from (C₁-C₆)alkoxy, halo, hydroxy and phenyl.

More preferably, R¹ is selected from phenyl and pyridyl, each of which is optionally substituted by one or more groups each independently selected from halo, (C₁-C₆)alkyl, (C₁-C₆)alkoxy, and cyano.

Yet more preferably, R¹ is selected from phenyl and pyridyl, each of which is optionally substituted by one or more groups each independently selected from fluoro, chloro, (C₁-C₃)alkyl, (C₁-C₃)alkoxy, and cyano.

Even more preferably, R¹ is selected from phenyl and pyridyl, each of which is optionally substituted by one or more groups each independently selected from fluoro, methyl, methoxy and cyano.

Most preferably, R¹ is selected from phenyl which is optionally substituted by one or two groups each independently selected from fluoro, methyl, methoxy and cyano, and pyridyl, which is substituted by methyl.

Preferably, R² is hydrogen, halo, CN, (C₁-C₃)alkyl, (C₁-C₃)alkoxy or NR⁹R¹⁰.

More preferably, R² is (C₁-C₃)alkoxy or NR⁹R¹⁰.

Yet more preferably, R² is methoxy or NHCH₃.

Most preferably, R² is methoxy.

R² is most preferably situated on the carbon adjacent to group Y.

Preferably, R³, R⁴, R⁵ and R⁶ are hydrogen.

Preferably, R⁸ is hydrogen, (C₁-C₆)alkyl, CO(C₁-C₆)alkyl, CO₂(C₁-C₆)alkyl, SO₂(C₁-C₆)alkyl or benzyl.

Most preferably, R⁸ is hydrogen, CH₃, COCH₃, CO₂CH₃, SO₂CH₃ or benzyl.

Most preferred compounds of formula (I) are:
8-Methoxy-1-(2′-methoxybiphenyl-4-yl)-4H,6H-[1,2,4]triazolo[4,3-a][4,1]benzoxazepine (Example 1),
8-Methoxy-1-(2′-methoxybiphenyl-4-yl)-5,6-dihydro-4H-pyrido[2,3-f][1,2,4]triazolo[4,3-a][1,4]diazepine (Example 4),
8-Methoxy-5-methyl-1-(2′-methoxybiphenyl-4-yl)-5,6-dihydro-4H-pyrido[2,3-f][1,2,4]triazolo[4,3-a][1,4]diazepine (Example 5),
5-Acetyl-8-methoxy-1-(2′-methoxybiphenyl-4-yl)-5,6-dihydro-4H-pyrido[2,3-f][1,2,4]triazolo[4,3-a][1,4]diazepine (Example 6),
8-Methoxy-1-(2′-methylbiphenyl-4-yl)-4H,6H-[1,2,4]triazolo[4,3-a][4,1]benzoxazepine (Example 9),
8-Methoxy-1-(2′-methylbiphenyl-4-yl)-5,6-dihydro-4H-pyrido[2,3-f][1,2,4]triazolo[4,3-a][1,4]diazepine (Example 12),
8-Methoxy-5-methyl-1-(2′-methylbiphenyl-4-yl)-5,6-dihydro-4H-pyrido[2,3-f][1,2,4]triazolo[4,3-a][1,4]diazepine (Example 13), and
5-Acetyl-8-methoxy-1-(2′-methylbiphenyl-4-yl)-5,6-dihydro-4H-pyrido[2,3-f][1,2,4]triazolo[4,3-a][1,4]diazepine (Example 14);
   and tautomers thereof and pharmaceutically acceptable salts, solvates and polymorphs of said compound or tautomer.

Pharmaceutically acceptable salts of the compounds of formula (I) include the acid addition and base salts thereof. Suitable acid addition salts are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts. For a review on suitable salts, see Handbook of Pharmaceutical Salts: Properties, Selection, and Use by Stahl and Wermuth (Wiley-VCH, Weinheim, Germany, 2002).

Pharmaceutically acceptable salts of compounds of formula (I) may be prepared by one or more of three methods:
(i) by reacting the compound of formula (I) with the desired acid or base;
(ii) by removing an acid- or base-labile protecting group from a suitable precursor of the compound of formula (I) or by ring-opening a suitable cyclic precursor, for example, a lactone or lactam, using the desired acid or base; or
(iii) by converting one salt of the compound of formula (I) to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of the invention may exist in both unsolvated and solvated forms. The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water.

Included within the scope of the invention are complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components which may be in stoichiometric or non-stoichiometric amounts. The resulting complexes may be ionised, partially ionised, or non-ionised. For a review of such complexes, see J Pharm Sci, 64 (8), 1269-1288, by Haleblian (August 1975). Hereinafter all references to compounds of formula (I) include references to salts, solvates and complexes thereof and to solvates and complexes of salts thereof.

The compounds of the invention include compounds of formula (I) as hereinbefore defined, including all polymorphs and crystal habits thereof, prodrugs and isomers thereof (including optical, geometric and tautomeric isomers) as hereinafter defined and isotopically-labeled compounds of formula (I).

As indicated, so-called ‘pro-drugs’ of the compounds of formula (I) are also within the scope of the invention. Thus certain derivatives of compounds of formula (I) which may have little or no pharmacological activity themselves can, when administered into or onto the body, be converted into compounds of formula (I) having the desired activity, for example, by hydrolytic cleavage. Such derivatives are referred to as ‘prodrugs’. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (ed. E. B. Roche, American Pharmaceutical Association).

Prodrugs in accordance with the invention can, for example, be produced by replacing appropriate functionalities present in the compounds of formula (I) with certain moieties known to those skilled in the art as ‘pro-moieties’ as described, for example, in Design of Prodrugs by H. Bundgaard (Elsevier, 1985).

Some examples of prodrugs in accordance with the invention include
(i) where the compound of formula (I) contains a carboxylic acid functionality (—COOH), an ester thereof, for example, a compound wherein the hydrogen of the carboxylic acid functionality of the compound of formula (I) is replaced by (C₁-C₈)alkyl;
(ii) where the compound of formula (I) contains an alcohol functionality (—OH), an ether thereof, for example, a compound wherein the hydrogen of the alcohol functionality of the compound of formula (I) is replaced by (C₁-C₆)alkanoyloxymethyl; and
(iii) where the compound of formula (I) contains a primary or secondary amino functionality (—NH₂ or —NHR where R≠H), an amide thereof, for example, a compound wherein, as the case may be, one or both hydrogens of the amino functionality of the compound of formula (I) is/are replaced by (C₁-C₁₀)alkanoyl.

Further examples of replacement groups in accordance with the foregoing examples and examples of other prodrug types may be found in the aforementioned references. Moreover, certain compounds of formula (I) may themselves act as prodrugs of other compounds of formula (I).

Also included within the scope of the invention are metabolites of compounds of formula (I), that is, compounds formed in vivo upon administration of the drug. Some examples of metabolites in accordance with the invention include
(i) where the compound of formula (I) contains a methyl group, an hydroxymethyl derivative thereof (—CH₃->—CH₂OH):
(ii) where the compound of formula (I) contains an alkoxy group, an hydroxy derivative thereof (—OR->—OH);
(iii) where the compound of formula (I) contains a tertiary amino group, a secondary amino derivative thereof (—NR¹R²->—NHR¹ or —NHR²);
(iv) where the compound of formula (I) contains a secondary amino group, a primary derivative thereof (—NHR¹->—NH₂);
(v) where the compound of formula (I) contains a phenyl moiety, a phenol derivative thereof (—Ph->—PhOH); and
(vi) where the compound of formula (I) contains an amide group, a carboxylic acid derivative thereof (—CONH₂->COOH).

Compounds of formula (I) containing one or more asymmetric carbon atoms can exist as two or more stereoisomers. Where a compound of formula (I) contains an alkenyl or alkenylene group, geometric cis/trans (or Z/E) isomers are possible. Where structural isomers are interconvertible via a low energy barrier, tautomeric isomerism (‘tautomerism’) can occur. This can take the form of proton tautomerism in compounds of formula (I) containing, for example, an imino, keto, or oxime group, or so-called valence tautomerism in compounds which contain an aromatic moiety. It follows that a single compound may exhibit more than one type of isomerism. Included within the scope of the present invention are all stereoisomers, geometric isomers and tautomeric forms of the compounds of formula (I), including compounds exhibiting more than one type of isomerism, and mixtures of one or more thereof. Also included are acid addition or base salts wherein the counterion is optically active, for example, d-lactate or l-lysine, or racemic, for example, dl-tartrate or dl-arginine. Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). Alternatively, the racemate (or a racemic precursor) may be reacted with a suitable optically active compound, for example, an alcohol, or, in the case where the compound of formula (I) contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture. Stereoisomeric conglomerates may be separated by conventional techniques known to those skilled in the art—see, for example, Stereochemistry of Organic Compounds by E. L. Eliel and S. H. Wilen (Wiley, N.Y., 1994).

The present invention includes all pharmaceutically acceptable isotopically-labelled compounds of formula (I) wherein one or more atoms are replaced by atoms having the same atomic number, but an atomic mass or mass number different from the atomic mass or mass number which predominates in nature. Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection. Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of formula (I) can generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

Also within the scope of the invention are intermediate compounds of formula (I) as hereinbefore defined, all salts, solvates and complexes thereof and all solvates and complexes of salts thereof as defined hereinbefore for compounds of formula (I). The invention includes all polymorphs of the aforementioned species and crystal habits thereof.

When preparing compounds of formula (I) in accordance with the invention, it is open to a person skilled in the art to routinely select the form of compound of formula (I) which provides the best combination of features for this purpose. Such features include the melting point, solubility, processability and yield of the intermediate form and the resulting ease with which the product may be purified on isolation.

Compounds of the invention intended for pharmaceutical use may be administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

They may be administered alone or in combination with one or more other compounds of the invention or in combination with one or more other drugs (or as any combination thereof). Generally, they will be administered as a formulation in association with one or more pharmaceutically acceptable excipients. The term ‘excipient’ is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Mack Publishing Company, 1995).

The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including
liquid-filled), chews, multi- and nano-particulates, gels, solid solution, liposome, films, ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986, by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate. Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet. Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet. Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant. Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated. The formulation of tablets is discussed in Pharmaceutical Dosage Forms: Tablets, Vol. 1, by H. Lieberman and L. Lachman (Marcel Dekker, N.Y., 1980).

Consumable oral films for human or veterinary use are typically pliable water-soluble or water-swellable thin film dosage forms which may be rapidly dissolving or mucoadhesive and typically comprise a compound of formula (I), a film-forming polymer, a binder, a solvent, a humectant, a plasticiser, a stabiliser or emulsifier, a viscosity-modifying agent and a solvent. Some components of the formulation may perform more than one function. The compound of formula (I) may be water-soluble or insoluble. A water-soluble compound typically comprises from 1 weight % to 80 weight %, more typically from 20 weight % to 50 weight %, of the solutes. Less soluble compounds may comprise a greater proportion of the composition, typically up to 88 weight % of the solutes. Alternatively, the compound of formula (I) may be in the form of multiparticulate beads. The film-forming polymer may be selected from natural polysaccharides, proteins, or synthetic hydrocolloids and is typically present in the range 0.01 to 99 weight %, more typically in the range 30 to 80 weight %. Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents. Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release. Suitable modified release formulations for the purposes of the invention are described in U.S. Pat. No. 6,106,864. Details of other suitable release technologies such as high energy dispersions and osmotic and coated particles are to be found in Pharmaceutical Technology On-line, 25(2), 1-14, by Verma et al (2001). The use of chewing gum to achieve controlled release is described in WO 00/35298.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilisation, may readily be accomplished using standard pharmaceutical techniques well known to those skilled in the art. The solubility of compounds of formula (I) used in the preparation of parenteral solutions may be increased by the use of appropriate formulation techniques, such as the incorporation of solubility-enhancing agents. Formulations for parenteral administration may be formulated to be immediate and/or modified release. Modified release formulations include delayed-, sustained-, pulsed-, controlled-, targeted and programmed release. Thus compounds of the invention may be formulated as a solid, semi-solid, or thixotropic liquid for administration as an implanted depot providing modified release of the active compound. Examples of such formulations include drug-coated stents and poly(dl-lactic-coglycolic)acid (PGLA) microspheres.

The compounds of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999). Other means of topical administration include delivery by electroporation, iontophoresis, phonophoresis, sonophoresis and microneedle or needle-free (e.g. Powderject™, Bioject™, etc.) injection. Formulations for topical administration may be formulated to be immediate and/or modified release. Modified release formulations include delayed-, sustained-, pulsed-, controlled-, targeted and programmed release.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin. The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying. Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of formula (I), propylene glycol, sterile water, ethanol and sodium chloride. Alternative solvents which may be used instead of propylene glycol include glycerol and polyethylene glycol. Suitable flavours, such as menthol and levomenthol, or sweeteners, such as saccharin or saccharin sodium, may be added to those formulations of the invention intended for inhaled/intranasal administration. Formulations for inhaled/intranasal administration may be formulated to be immediate and/or modified release using, for example, PGLA. Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing from 2 to 30 mg of the compound of formula (I). The overall daily dose will typically be in the range 50 to 100 mg which may be administered in a single dose or, more usually, as divided doses throughout the day.

The compounds of the invention may be administered rectally or vaginally, for example, in the form of a suppository, pessary, or enema. Cocoa butter is a traditional suppository base, but various alternatives may be used as appropriate. Formulations for rectal/vaginal administration may be formulated to be immediate and/or modified release. Modified release formulations include delayed-, sustained-, pulsed-, controlled-, targeted and programmed release.

The compounds of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis. Formulations for ocular/aural administration may be formulated to be immediate and/or modified release. Modified release formulations include delayed-, sustained-, pulsed-, controlled-, targeted, or programmed release.

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration. Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

Inasmuch as it may desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for coadministration of the compositions. Thus the kit of the invention comprises two or more separate pharmaceutical compositions, at least one of which contains a compound of formula (I) in accordance with the invention, and means for separately retaining said compositions, such as a container, divided bottle, or divided foil packet. An example of such a kit is the familiar blister pack used for the packaging of tablets, capsules and the like. The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

For administration to human patients, the total daily dose of the compounds of the invention is typically in the range 50 mg to 100 mg depending, of course, on the mode of administration and efficacy. For example, oral administration may require a total daily dose of from 50 mg to 100 mg. The total daily dose may be administered in single or divided doses and may, at the physician's discretion, fall outside of the typical range given herein. These dosages are based on an average human subject having a weight of about 60 kg to 70 kg. The physician will readily be able to determine doses for subjects whose weight falls outside this range, such as infants and the elderly.

For the avoidance of doubt, references herein to “treatment” include references to curative, palliative and prophylactic treatment.

Processes

Compounds of the invention may be prepared, in known manner in a variety of ways. In the following reaction schemes and hereafter, unless otherwise stated U, V, W, Z, Y, R¹, R², R³, R⁴, R⁵ and R⁶ are as defined in the first aspect. These processes form further aspects of the invention.

In one aspect, the present invention comprises a process for the preparation of a compound of formula (I) where X is O and U, V, W, Z, Y, R¹, R², R³, R⁴, R⁵, and R⁶ are described herein may be prepared according to reaction scheme 1.

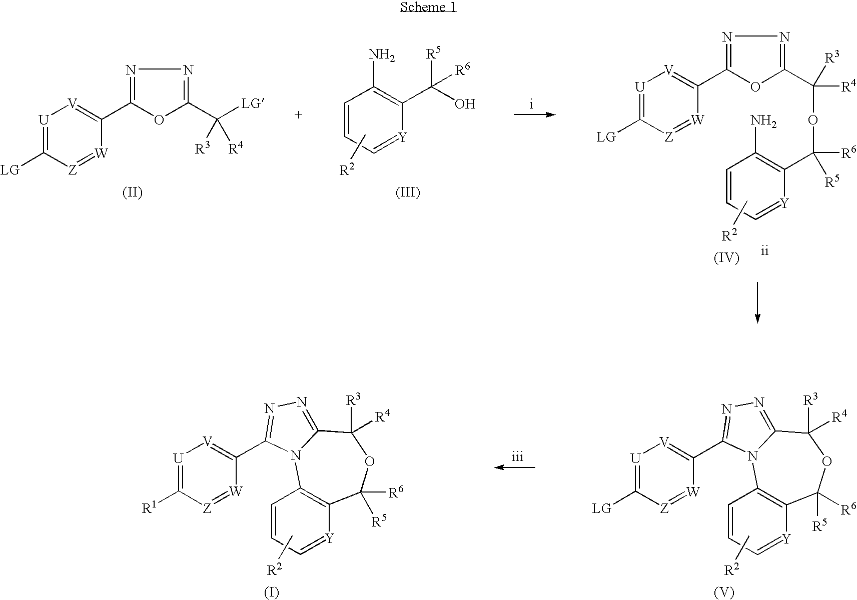

LG is a leaving group, typically a halo and preferably bromo. LG′ is a leaving group such as halo or mesylate and is preferably chloro.

Compounds of formula (II) are prepared as described in scheme 2. When Y═CH, compounds of formula (III) are either known in the literature or can be prepared using standard methodology: for example reduction of aryl esters, and hydrogenation of nitrobenzenes/nitropyridines. Alternatively, when Y═N or CR⁷, compounds of formula (III) can be prepared as described in scheme 3.

Compounds of formula (IV) can be prepared from the compounds of formula (II) by process step (i), which comprises reaction with an excess of suitable alcohol of formula (III) in the presence of a suitable base such as sodium hydride, or n-butyl lithium, in a suitable solvent such as tetrahydrofuran or toluene at low temperature. Typical conditions comprise reaction of 1.0 equivalent of compound (II), 1.5 to 2.0 equivalents of compound (III) and 1.5 to 2.0 equivalents of sodium hydride in tetrahydrofuran at −10° C. for 2 hours.

Compounds of formula (V) can be prepared from compounds of formula (IV) by process step (ii), which comprises heating compound (IV) to elevated temperatures, such as 70 to 150° C., for 1 to 48 hours in the presence of a suitable acidic catalyst such as para-toluenesulfonic acid or trifluoroacetic acid, in a suitable high boiling solvent such as xylene or toluene. Typical conditions comprise reaction of 1.0 equivalent of compound (IV) and catalytic para-toluenesulfonic acid in toluene at 80° C. for 5 hours.

Compounds of formula (I) can be prepared from compounds of formula (V) by process step (iii), which comprises a Suzuki coupling by reaction with a suitable boronic acid such as methoxybenzeneboronic acid (available commercially), in a suitable solvent such as 1,2-dimethoxyethane, or 1,4-dioxane, in the presence of a suitable base such as sodium carbonate or caesium carbonate, and a suitable palladium catalyst as described in the literature: Suzuki, A Pure & Appl. Chem. 1985, 57, 1749 and references contained within. Typical conditions comprise reaction of 1.0 equivalent of aryl bromide (V), 1.5 to 2.5 equivalents of boronic acid, 2.0 to 3.0 equivalents of sodium carbonate, and catalytic tetrakis-(triphenylphosphine) palladium in wet 1,2-dimethoxyethane heated between 100 to 150° C. for 1 to 3 hours.

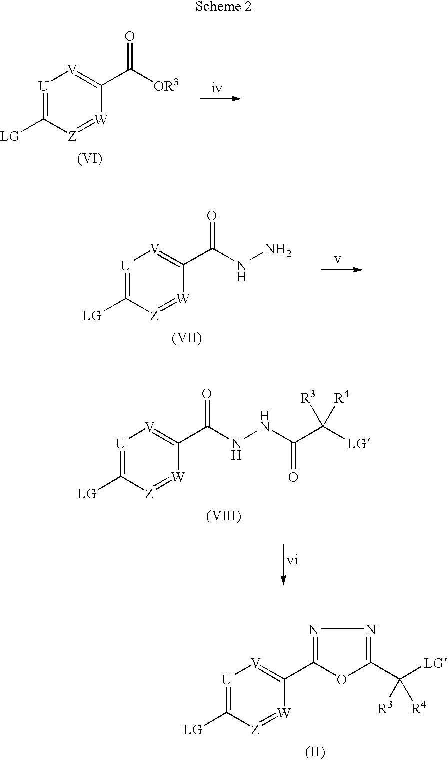

LG is a leaving group, typically halo, preferably bromo.

Compounds of general formula (VI) are commercially available. When W is N, compounds of formula (VI) can also be prepared by analogy with the method of J. J. Song and N. K. Yee (J. Org. Chem. 2001, 66, 605-608).

Compounds of formula (VII) can be prepared from compounds of formula (VI) by process step (iv), which comprises reaction with an excess of hydrazine monohydrate in a suitable solvent such as methanol or ethanol at reflux. Typical conditions comprise reaction of 1.0 equivalent of aryl ester (VI) and 3 equivalents of hydrazine monohydrate in methanol heated to 75° C. for 48 hours.

Compounds of general formula (VIII) can be prepared from compound (VII) by process step (v), which comprises reaction with an acid chloride LG′CR₃R₄C(O)Cl in the presence of a base such as triethylamine, N-methylmorpholine, sodium carbonate or potassium hydroxide, in a suitable solvent such as dichloromethane or tetrahydrofuran at ambient temperature. Typical conditions comprise reaction of 1.0 equivalents of aryl hydrazide (VII), 1.0 to 1.3 equivalents of acid chloride LG′CR₃R₄C(O)Cl and 1.2 to 2.0 equivalents of N-methylmorpholine in dichloromethane at 25° C.

LG′ is a leaving group such as halo or mesylate and is preferably chloro.

Compounds of formula (II) can be prepared from diacylhydrazines of formula (VIII) by process step (vi), which comprises reaction with a suitable dehydrating agent such as phosphorous oxychloride, trifluoromethanesulfonic anhydride, or phosphorous pentachloride, optionally in the presence of a base such as pyridine and a suitable solvent such as dichloromethane or acetonitrile, at temperatures between 25° C. and 110° C. Typical conditions comprise reaction of 1.0 equivalents of diacylhydrazine (VIII) in phosphorous oxychloride at 110° C. for 4 hours.

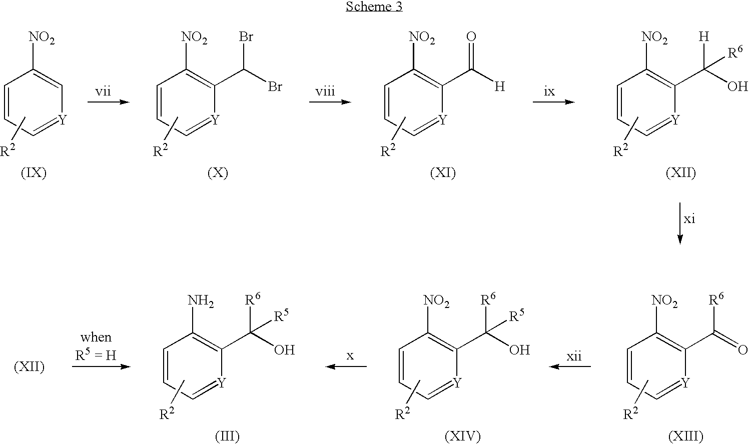

Compounds of general formula (IX) are commercially available.

Compounds of general formula (X) can be prepared by process step (vii) as described in the literature: J. Org Chem. 54(21), 5094-5100; 1989. Typical conditions comprise reaction of 1.0 equivalent of compound (IX), 1.0-2.0 equivalents of bromoform and 3.0-5.0 equivalent of potassium tert-butoxide in tetrahydrofuran at −73° C. for 2 hours.

Compounds of general formula (XI) can be prepared from compounds of formula (X) by process step (viii) using an analogous method to that of R. A. Daines et al (J. Med Chem. 36(22), 3321-3332; 1993). Typical conditions comprise reaction of 1.0 equivalent of compound (X) and 2.0-2.5 equivalents of silver nitrate in aqueous ethanol, heated under reflux for 5 hours.

Compounds of general formula (XII) can be prepared from compounds of general formula (XI) by process step (ix), which comprises reaction with an organometallic agent such as sodium borohydride, lithium aluminium hydride, diisobutylammonium hydride, R⁵MgBr, and R⁵Li in a suitable solvent such as tetrahydrofuran, diethyl ether or N,N-dimethylformamide, stirring at a temperature between 0 to 120° C. for 1 to 5 hours. Typical conditions comprise reaction of 1.0 equivalent of compound (XI) and 1.0-4.0 equivalents of sodium borohydride in tetrahydrofuran at 0° C. to room temperature for 30 minutes.

Compounds of general formula (XIV) where R⁵ and R⁶≠H can be prepared from compounds of general formula (XIII) by process step (xii)—reaction with an organometallic agent such as R⁵MgBr or R⁵Li in a suitable solvent such as tetrahydrofuran, diethyl ether or N,N-dimethylformamide, stirring at a temperature between 0-120° C. for 1-5 hours. Typical conditions comprise reaction of 1.0 equivalent of compound (XIII) and 1.0-4.0 equivalents of MeMgBr in tetrahydrofuran at 0° C. to room temperature. Compounds of general formula (XIII) can be prepared from compounds of general formula (XII) by process step (xi), which comprises oxidation with a suitable reagent such as CrO₃ or SO₃.pyridine in a suitable solvent such as dichloromethane or diethyl ether stirring at a temperature between −78° C. and room temperature. Typical conditions comprise reaction of 1.0 equivalent of compound (XII) and 2.0 equivalents of SO₃.pyridine in dichloromethane at room temperature.

Compounds of general formula (III) can be prepared from compounds of general formula (XII) and (XIV) by process step (x), which comprises hydrogenation in the presence of a suitable catalyst such as 10% Pd/C or Raney nickel® in a suitable solvent such as ethanol or methanol. Typical conditions comprise reaction of 1.0 equivalent of compound (XII) and 10% Pd/C (cat.) in ethanol at room temperature, under 414 kPa (60 psi) of hydrogen, for 1 hour.

Compounds of general formula (V) where X is O and R¹, R², R³, R⁴, R⁵, R⁶, U, V, W, Z, and Y are as described herein may be alternatively prepared according to reaction scheme 4.

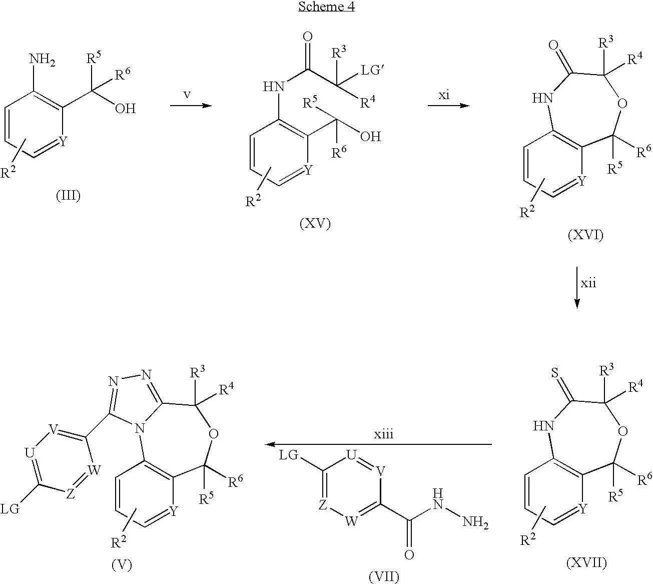

Compounds of formula (XV) can be prepared from compound (III) by process step (v) as described in scheme 2. Compound (XVI) can be prepared from compound (XV) by process step (xi) as described in the literature: J. Org Chem. 51(25), 5001-2; 1986. Typical conditions comprise reaction of 1.0 equivalent of compound (XIII) and 2.0-2.5 equivalents of potassium tert-butoxide in tert-butanol at 25° C. for 15 minutes.

Compound (XVII) can be prepared from compound (XVI) by process step (xii), which comprises reaction with a suitable thionating agent such Lawesson's reagent or phosphorous pentasulfide, optionally in the presence of a base such as sodium carbonate, in a suitable solvent such as tetrahydrofuran at a temperature between 0-25° C. Typical conditions comprise reaction of 1.0 equivalent of compound (XVI), 1.0-1.5 equivalents of phosphorous pentasulfide and 1.0-1.5 equivalents of sodium carbonate in tetrahydrofuran at 25° C. for 3 hours.

Compounds of general formula (VII) can be prepared by process step (iv) as described in scheme 2. Compounds of general formula (V) can be prepared from thioamide compound (XVII) by process step (xiii), which comprises reaction with hydrazide compound (VII) in a suitable solvent such as ethanol or n-butanol at elevated temperature. Typical conditions comprise reaction of 1.0 equivalent of thioamide (XVII), 1.0-2.0 equivalents of hydrazide (VII) in n-butanol at reflux for 10 hours.

Compounds of general formula (I) where X is NR⁵ and U, V, W, Z, Y, R¹, R², R³, R⁴, and R⁵ are as described herein may be alternatively prepared according to reaction scheme 5.

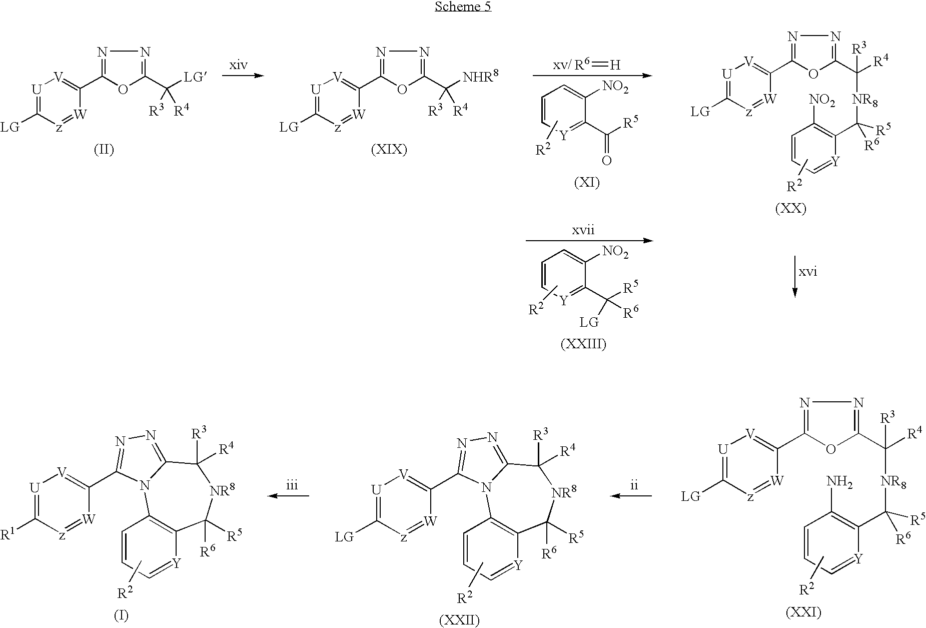

Compounds of general formula (II) can be prepared as described previously in scheme 2.

Compounds of general formula (XIX) can be prepared from compound (II) by process step (xiv), which comprises reaction with a suitable amine NH₂R⁸ optionally in the presence of a suitable base such as potassium carbonate, sodium carbonate or cesium carbonate, in a suitable solvent such as acetonitrile or N,N-dimethylformamide heating between 25-70° C. for 2-18 hours. Typical conditions comprise reaction of 1.0 equivalent of (II) where LG=chloro, 1.0-1.5 equivalents of amine NH₂R⁸ and 2.0 equivalents of potassium carbonate in acetonitrile at 60° C. for 6 hours.

Compounds of general formula (XX) where R⁶=H can be prepared from compounds of formula (XIX) by process step (xv), which comprises reaction with aldehyde or ketone (XI) in the presence of a reducing agent such as sodium triacetoxyborohydride or sodium cyanoborohydride in a suitable solvent such as dichloromethane or tetrahydrofuran at 0-50° C. Typical conditions comprise reaction of 1.0 equivalent of compound (XIX), 1.0-1.5 equivalents of compound (XI) and sodium triacetoxyborohydride in dichloromethane, at 25° C. for 6 hours.

Compounds of general formula (XX) can be prepared from compounds of formula (XIX) by process step (xvii), which comprises reaction with a compound of formula (XXIII) such as where LG is Cl or Br in a suitable solvent such as dichloromethane or dimethylformamide in the presence of a suitable base such as potassium carbonate or sodium hydride.

Compounds of general formula (XXI) can be prepared from compounds of formula (XX) by process step (xvi), which comprises reduction of the nitro group by heating compound (XX) to elevated temperatures in the presence of a suitable reactive metal such as iron, tin or zinc in a suitable acid such as acetic acid or hydrochloric acid. Typical conditions comprise of 1.0 equivalent of nitro compound (XX) and 2.0-3.0 equivalents of iron powder in acetic acid at 60° C. for 3 hours. Alternatively, compounds of general formula (XXI) can be prepared from compounds of formula (XX) by process step (x) as described previously in scheme 3.

Compounds of general formula (XXII) can be prepared from compounds of general formula (XXI) by process step (ii) as described in scheme 1.

Compounds of general formula (I) can be prepared from compounds of formula (XXII) by process step (iii) as described in scheme 1.

Compounds of formula (I) may also be converted to alternative compounds of formula (I) using standard chemical reactions and transformations. For example when X is NR⁸and R³ represents benzyl, a series of amines, amides and sulfonamides can be prepared by deprotection and subsequent derivatisation of the amino functional group. This is exemplified in examples 5-8 and 13-16.

All of the above reactions and the preparations of novel starting materials disclosed in the preceding methods are conventional and appropriate reagents and reaction conditions for their performance or preparation as well as procedures for isolating the desired products will be well known to those skilled in the art with reference to literature precedents and the examples and preparations hereto.

The compounds of the invention are useful because they have pharmacological activity in mammals, including humans. More particularly, they are useful in the treatment or prevention of a disorder in which modulation of the levels of oxytocin could provide a beneficial effect. Disease states that may be mentioned include sexual dysfunction, particularly premature ejaculation, preterm labour, complications in labour, appetite and feeding disorders, benign prostatic hyperplasia, premature birth, dysmenorrhoea (primary and secondary), congestive heart failure, arterial hypertension, liver cirrhosis, nephrotic hypertension, occular hypertension, obsessive compulsive disorder and neuropsychiatric disorders.

The compounds of the invention are also useful in the treatment or prevention of anxiety, cardiovascular disease (including angina, atherosclerosis, hypertension, heart failure, edema, hypernatremia), inappropriate secretion of vasopressin, endometriosis, emesis (including motion sickness), intrauterine growth retardation, inflammation (including rheumatoid arthritis), mittlesmerchz, preclampsia, premature ejaculation, premature (preterm) labor and Raynaud's disease.

Sexual dysfunction (SD) is a significant clinical problem which can affect both males and females. The causes of SD may be both organic as well as psychological. Organic aspects of SD are typically caused by underlying vascular diseases, such as those associated with hypertension or diabetes mellitus, by prescription medication and/or by psychiatric disease such as depression. Physiological factors include fear, performance anxiety and interpersonal conflict. SD impairs sexual performance, diminishes self-esteem and disrupts personal relationships thereby inducing personal distress. In the clinic, SD disorders have been divided into female sexual dysfunction (FSD) disorders and male sexual dysfunction (MSD) disorders (Melman et al, J. Urology, 1999, 161, 5-11).

FSD can be defined as the difficulty or inability of a woman to find satisfaction in sexual expression. FSD is a collective term for several diverse female sexual disorders (Leiblum, S. R. (1998). Definition and classification of female sexual disorders. Int. J. Impotence Res., 10, S104-S106; Berman, J. R., Berman, L. & Goldstein, I. (1999). Female sexual dysfunction: Incidence, pathophysiology, evaluations and treatment options. Urology, 54, 385-391). The woman may have lack of desire, difficulty with arousal or orgasm, pain with intercourse or a combination of these problems. Several types of disease, medications, injuries or psychological problems can cause FSD. Treatments in development are targeted to treat specific subtypes of FSD, predominantly desire and arousal disorders.

The categories of FSD are best defined by contrasting them to the phases of normal female sexual response: desire, arousal and orgasm (Leiblum, S. R. (1998). Definition and classification of female sexual disorders, Int. J. Impotence Res., 10, S104-S106). Desire or libido is the drive for sexual expression. Its manifestations often include sexual thoughts either when in the company of an interested partner or when exposed to other erotic stimuli. Arousal is the vascular response to sexual stimulation, an important component of which is genital engorgement and includes increased vaginal lubrication, elongation of the vagina and increased genital sensation/sensitivity. Orgasm is the release of sexual tension that has culminated during arousal.

Hence, FSD occurs when a woman has an inadequate or unsatisfactory response in any of these phases, usually desire, arousal or orgasm. FSD categories include hypoactive sexual desire disorder, sexual arousal disorder, orgasmic disorders and sexual pain disorders. Although the compounds of the invention will improve the genital response to sexual stimulation (as in female sexual arousal disorder), in doing so it may also improve the associated pain, distress and discomfort associated with intercourse and so treat other female sexual disorders.

Thus, in accordance with a further aspect of the invention, there is provided the use of a compound of the invention in the preparation of a medicament for the treatment or prophylaxis of hypoactive sexual desire disorder, sexual arousal disorder, orgasmic disorder and sexual pain disorder, more preferably for the treatment or prophylaxis of sexual arousal disorder, orgasmic disorder, and sexual pain disorder, and most preferably in the treatment or prophylaxis of sexual arousal disorder. Hypoactive sexual desire disorder is present if a woman has no or little desire to be sexual, and has no or few sexual thoughts or fantasies. This type of FSD can be caused by low testosterone levels, due either to natural menopause or to surgical menopause. Other causes include illness, medications, fatigue, depression and anxiety.

Female sexual arousal disorder (FSAD) is characterised by inadequate genital response to sexual stimulation. The genitalia do not undergo the engorgement that characterises normal sexual arousal. The vaginal walls are poorly lubricated, so that intercourse is painful. Orgasms may be impeded. Arousal disorder can be caused by reduced oestrogen at menopause or after childbirth and during lactation, as well as by illnesses, with vascular components such as diabetes and atherosclerosis. Other causes result from treatment with diuretics, antihistamines, antidepressants eg SSRIs or antihypertensive agents. Sexual pain disorders (includes dyspareunia and vaginismus) is characterised by pain resulting from penetration and may be caused by medications which reduce lubrication, endometriosis, pelvic inflammatory disease, inflammatory bowel disease or urinary tract problems.

The prevalence of FSD is difficult to gauge because the term covers several types of problem, some of which are difficult to measure, and because the interest in treating FSD is relatively recent. Many women's sexual problems are associated either directly with the female ageing process or with chronic illnesses such as diabetes and hypertension.

Because FSD consists of several subtypes that express symptoms in separate phases of the sexual response cycle, there is not a single therapy. Current treatment of FSD focuses principally on psychological or relationship issues. Treatment of FSD is gradually evolving as more clinical and basic science studies are dedicated to the investigation of this medical problem. Female sexual complaints are not all psychological in pathophysiology, especially for those individuals who may have a component of vasculogenic dysfunction (eg FSAD) contributing to the overall female sexual complaint. There are at present no drugs licensed for the treatment of FSD. Empirical drug therapy includes oestrogen administration (topically or as hormone replacement therapy), androgens or mood-altering drugs such as buspirone or trazodone. These treatment options are often unsatisfactory due to low efficacy or unacceptable side effects.

The Diagnostic and Statistical Manual (DSM) IV of the American Psychiatric Association defines Female Sexual Arousal Disorder (FSAD) as being:
“a persistent or recurrent inability to attain or to maintain until completion of the sexual activity adequate lubrication-swelling response of sexual excitement. The disturbance must cause marked distress or interpersonal difficulty.”

The arousal response consists of vasocongestion in the pelvis, vaginal lubrication and expansion and swelling of the external genitalia. The disturbance causes marked distress and/or interpersonal difficulty.

FSAD is a highly prevalent sexual disorder affecting pre-, per- and post menopausal (±HRT) women. It is associated with concomitant disorders such as depression, cardiovascular diseases, diabetes and UG disorders. The primary consequences of FSAD are lack of engorgement/swelling, lack of lubrication and lack of pleasurable genital sensation. The secondary consequences of FSAD are reduced sexual desire, pain during intercourse and difficulty in achieving an orgasm.

Male sexual dysfunction (MSD) is generally associated with either erectile dysfunction, also known as male erectile dysfunction (MED) and/or ejaculatory disorders such as premature ejaculation, anorgasmia (unable to achieve orgasm) or desire disorders such as hypoactive sexual desire disorder (lack of interest in sex).

PE is a relatively common sexual dysfunction in men. It has been defined in several different ways but the most widely accepted is the Diagnostic and Statistical Manual of Mental Disorders IV one which states:
“PE is a lifelong persistent or recurrent ejaculation with minimal sexual stimulation before, upon or shortly after penetration and before the patient wishes it. The clinician must take into account factors that affect duration of the excitement phase, such as age, novelty of the sexual partner or stimulation, and frequency of sexual activity. The disturbance causes marked distress of interpersonal difficulty.”

The International Classification of Diseases 10 definition states:
“There is an inability to delay ejaculation sufficiently to enjoy lovemaking, manifest as either of the following: (1) occurrence of ejaculation before or very soon after the beginning of intercourse (if a time limit is required: before or within 15 seconds of the beginning of intercourse); (2) ejaculation occurs in the absence of sufficient erection to make intercourse possible. The problem is not the result of prolonged abstinence from sexual activity”

Other definitions which have been used include classification on the following criteria:
Related to partner's orgasm
Duration between penetration and ejaculation
Number of thrust and capacity for voluntary control

Psychological factors may be involved in PE, with relationship problems, anxiety, depression, prior sexual failure all playing a role.

Ejaculation is dependent on the sympathetic and parasympathetic nervous systems. Efferent impulses via the sympathetic nervous system to the vas deferens and the epididymis produce smooth muscle contraction, moving sperm into the posterior urethra. Similar contractions of the seminal vesicles, prostatic glands and the bulbouretheral glands increase the volume and fluid content of semen. Expulsion of semen is mediated by efferent impulses originating from a population of lumber spinothalamic cells in the lumbosacral spinal cord (Coolen & Truitt, Science, 2002, 297, 1566) which pass via the parasympathetic nervous system and cause rhythmic contractions of the bulbocavernous, ischiocavernous and pelvic floor muscles. Cortical control of ejaculation is still under debate in humans. In the rat the medial pre-optic area and the paraventricular nucleus of the hypothalamus seem to be involved in ejaculation.

Ejaculation comprises two separate components—emission and ejaculation. Emission is the deposition of seminal fluid and sperm from the distal epididymis, vas deferens, seminal vesicles and prostrate into the prostatic urethra. Subsequent to this deposition is the forcible expulsion of the seminal contents from the urethral meatus. Ejaculation is distinct from orgasm, which is purely a cerebral event. Often the two processes are coincidental.

A pulse of oxytocin in peripheral serum accompanies ejaculation in mammals. In man oxytocin but not vasopressin plasma concentrations are significantly raised at or around ejaculation. Oxytocin does not induce ejaculation itself; this process is 100% under nervous control via α1-adrenoceptor/sympathetic nerves originating from the lumbar region of the spinal cord. The systemic pulse of oxytocin may have a role in the peripheral ejaculatory response. It could serve to modulate the contraction of ducts and glandular lobules throughout the male genital tract, thus influencing the fluid volume of different ejaculate components for example. Oxytocin released centrally into the brain could influence sexual behaviour, subjective appreciation of arousal (orgasm) and latency to subsequent ejaculation. Accordingly, one aspect of the invention provides for the use of a compound of formula (I) in the preparation of a medicament for the prevention or treatment of sexual dysfunction, preferably male sexual dysfunction, most preferably premature ejaculation.

It has been demonstrated in the scientific literature that the number of oxytocin receptors in the uterus increases during pregnancy, most markedly before the onset of labour (Gimpl & Fahrenholz, 2001, Physiological Reviews, 81 (2), 629-683.). Without being bound by any theory it is known that the inhibition of oxytocin can assist in preventing preterm labour and in resolving complications in labour. Accordingly, another aspect of the invention provides for the use of a compound of formula (I) in the preparation of a medicament for the prevention or treatment of preterm labour and complications in labour.

Oxytocin has a role in feeding; it reduces the desire to eat (Arletti et al., Peptides, 1989, 10, 89). By inhibiting oxytocin it is possible to increase the desire to eat. Accordingly oxytocin inhibitors are useful in treating appetite and feeding disorders. Accordingly, a further aspect of the invention provides for the use of a compound of formula (I) in the preparation of a medicament for the prevention or treatment of appetite and feeding disorders.

Oxytocin is implicated as one of the causes of benign prostatic hyperplasia (BPH). Analysis of prostate tissue have shown that patients with BPH have increased levels of oxytocin (Nicholson & Jenkin, Adv. Exp. Med. & Biol., 1995, 395, 529). Oxytocin antagonists can help treat this condition. Accordingly, another aspect of the invention provides for the use of a compound of formula (I) in the preparation of a medicament for the prevention or treatment of benign prostatic hyperplasia.

Oxytocin has a role in the causes of dysmenorrhoea due to its activity as a uterine vasoconstrictor (Akerlund, Ann. NY Acad. Sci., 1994, 734, 47). Oxytocin antagonists can have a therapeutic effect on this condition. Accordingly, a further aspect of the invention provides for the use of a compound of formula (I) in the preparation of a medicament for the prevention of treatment of dysmenorrhoea.

It is to be appreciated that all references herein to treatment include curative, palliative and prophylactic treatment.

The compounds of the present invention may be coadministered with one or more agents selected from:
1) One or more selective serotonin reuptake inhibitors (SSRIs) such as dapoxetine, paroxetine, 3-[(dimethylamino)methyl]-4-[4-(methylsulfanyl)phenoxy]benzenesulfonamide (Example 28, WO 0172687), 3-[(dimethylamino)methyl]-4-[3-methyl-4-(methylsulfanyl)phenoxy]benzenesulfonamide (Example 12, WO 0218333), N-methyl-N-({3-[3-methyl-4-(methylsulfanyl)phenoxy]-4-pyridinyl}methyl)amine (Example 38, PCT Application no PCT/IB02/01032).
2) One or more local anaesthetics;
3) one or more α-adrenergic receptor antagonists (also known as α-adrenoceptor blockers, α-receptor blockers or α-blockers); suitable α₁-adrenergic receptor antagonists include: phentolamine, prazosin, phentolamine mesylate, trazodone, alfuzosin, indoramin, naftopidil, tamsulosin, phenoxybenzamine, rauwolfa alkaloids, Recordati 15/2739, SNAP 1069, SNAP 5089, RS17053, SL 89.0591, doxazosin, Example 19 of W09830560, terazosin and abanoquil; suitable α₂-adrenergic receptor antagonists include dibenarnine, tolazoline, trimazosin, efaroxan, yohimbine, idazoxan clonidine and dibenarnine; suitable non-selective α-adrenergic receptor antagonists include dapiprazole; further α-adrenergic receptor antagonists are described in PCT application WO99/30697 published on 14 Jun. 1998 and U.S. Pat. Nos. 4,188,390; 4,026,894; 3,511,836; 4,315,007; 3,527,761; 3,997,666; 2,503,059; 4,703,063; 3,381,009; 4,252,721 and 2,599,000 each of which is incorporated herein by reference;
4) one or more cholesterol lowering agents such as statins (e.g. atorvastatin/Lipitor—trade mark) and fibrates;
5) one or more of a serotonin receptor agonist, antagonist or modulator, more particularly agonists, antagonists or modulators for example 5HT1A, 5HT2A, 5HT2C, 5HT3, 5HT6 and/or 5HT7 receptors, including those described in WO-09902159, WO-00002550 and/or WO-00028993;
6) one or more NEP inhibitors, preferably wherein said NEP is EC 3.4.24.11 and more preferably wherein said NEP inhibitor is a selective inhibitor for EC 3.4.24.11, more preferably a selective NEP inhibitor is a selective inhibitor for EC 3.4.24.11, which has an IC₅₀ of less than 100 nM (e.g. ompatrilat, sampatrilat) suitable NEP inhibitor compounds are described in EP-A-1097719; IC₅₀ values against NEP and ACE may be determined using methods described in published patent application EP1097719-A1, paragraphs [0368] to [0376];
7) one or more of an antagonist or modulator for vasopressin receptors, such as relcovaptan (SR 49059), conivaptan, atosiban, VPA-985, CL-385004, Vasotocin.
8) Apomorphine—teachings on the use of apomorphine as a pharmaceutical may be found in U.S. Pat. No. 5,945,117;
9) Dopamine agonists (in particular selective D2, selective D3, selective D4 and selective D2-like agents) such as Pramipexole (Pharmacia Upjohn compound number PNU95666), ropinirole, apomorphine, surmanirole, quinelorane, PNU-142774, bromocriptine, carbergoline, Lisuride;
10) Melanocortin receptor agonists (e.g. Melanotan II and PT141) and selective MC3 and MC4 agonists (e.g. THIQ);
11) Mono amine transport inhibitors, particularly Noradrenaline Re-uptake Inhibitors (NRIs) (e.g. Reboxetine), other Serotonin Re-uptake Inhibitors (SRIs) (e.g. paroxetine, dapoxetine) or Dopamine Re-uptake Inhibitors (DRIs);
12) 5-HT_{1A} antagonists (e.g. robalzotan); and
13) PDE inhibitors such as PDE2 (e.g. erythro-9-(2-hydroxyl-3-nonyl)-adenine) and Example 100 of EP 0771799-incorporated herein by reference) and in particular a PDE5 inhibitor such as the pyrazolo[4,3-d]pyrimidin-7-ones disclosed in EP-A-0463756; the pyrazolo[4,3-d]pyrimidin-7-ones disclosed in EP-A-0526004; the pyrazolo[4,3-d]pyrimidin-7-ones disclosed in published international patent application WO 93/06104; the isomeric pyrazolo[3,4-d]pyrimidin-4-ones disclosed in published international patent application WO 93/07149; the quinazolin-4-ones disclosed in published international patent application WO 93/12095; the pyrido[3,2-d]pyrimidin-4-ones disclosed in published international patent application WO 94/05661; the purin-6-ones disclosed in published international patent application WO 94/00453; the pyrazolo[4,3-d]pyrimidin-7-ones disclosed in published international patent application WO 98/49166; the pyrazolo[4,3-d]pyrimidin-7-ones disclosed in published international patent application WO 99/54333; the pyrazolo[4,3-d]pyrimidin-4-ones disclosed in EP-A-0995751; the pyrazolo[4,3-d]pyrimidin-7-ones disclosed in published international patent application WO 00/24745; the pyrazolo[4,3-d]pyrimidin-4-ones disclosed in EP-A-0995750; the compounds disclosed in published international application W095/19978; the compounds disclosed in published international application WO 99/24433 and the compounds disclosed in published international application WO 93/07124. The pyrazolo[4,3-d]pyrimidin-7-ones disclosed in published international application WO 01/27112; the pyrazolo[4,3-d]pyrimidin-7-ones disclosed in published international application WO 01/27113; the compounds disclosed in EP-A-1092718 and the compounds disclosed in EP-A-1092719.

Preferred PDE5 inhibitors for use with the invention:
5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil) also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methylpiperazine (see EP-A-0463756);
5-(2-ethoxy-5-morpholinoacetylphenyl)-1-methyl-3-n-propyl-1,6-dihydro-7H-pyrazolo[4,3-d]pyrimidin-7-one (see EP-A-0526004);
3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one (see WO98/49166);
3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one (see WO99/54333);
(+)-3-ethyl-5-[5-(4-ethylpiperazin-1-ylsulphonyl)-2-(2-methoxy-1(R)-methylethoxy)pyridin-3-yl]-2-methyl-2,6-dihydro-7H-pyrazolo[4,3-d]pyrimidin-7-one, also known as 3-ethyl-5-{5-[4-ethylpiperazin-1-ylsulphonyl]-2-([(1R)-2-methoxy-1-methylethyl]oxy)pyridin-3-yl}-2-methyl-2,6-dihydro-7H-pyrazolo[4,3-d]pyrimidin-7-one (see WO99/54333);
5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, also known as 1-{6-ethoxy-5-[3-ethyl-6,7-dihydro-2-(2-methoxyethyl)-7-oxo-2H-pyrazolo[4,3-d]pyrimidin-5-yl]-3-pyridylsulphonyl}-4-ethylpiperazine (see WO 01/27113, Example 8);
5-[2-iso-Butoxy-5-(4-ethylpiperazin-1-ylsulphonyl)pyridin-3-yl]-3-ethyl-2-(1-methylpiperidin-4-yl)-2,6-dihydro-7H-pyrazolo[4,3-d]pyrimidin-7-one (see WO 01/27113, Example 15);
5-[2-Ethoxy-5-(4-ethylpiperazin-1-ylsulphonyl)pyridin-3-yl]-3-ethyl-2-phenyl-2,6-dihydro-7H-pyrazolo[4,3-d]pyrimidin-7-one (see WO 01/27113, Example 66);
5-(5-Acetyl-2-propoxy-3-pyridinyl)-3-ethyl-2-(1-isopropyl-3-azetidinyl)-2,6-dihydro-7H-pyrazolo[4,3-d]pyrimidin-7-one (see WO 01/27112, Example 124);
5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one (see WO 01/27112, Example 132);
(6R,12aR)-2,3,6,7,12,12a-hexahydro-2-methyl-6-(3,4-methylenedioxyphenyl)-pyrazino[2′,1′:6,1]pyrido[3,4-b]indole-1,4-dione (IC-351), i.e. the compound of examples 78 and 95 of published international application W095/19978, as well as the compound of examples 1, 3, 7 and 8;
2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil) also known as 1-[[3-(3,4-dihydro-5-methyl-4-oxo-7-propylimidazo[5,1-f]-as-triazin-2-yl)-4-ethoxyphenyl]sulphonyl]-4-ethylpiperazine, i.e. the compound of examples 20, 19, 337 and 336 of published international application WO99/24433; and
   the compound of example 11 of published international application WO93/07124 (EISAI); and compounds 3 and 14 from Rotella D P, J. Med. Chem., 2000, 43, 1257.

Still further PDE5 inhibitors for use with the invention include:
4-bromo-5-(pyridylmethylamino)-6-[3-(4-chlorophenyl)-propoxy]-3(2H)pyridazinone; 1-[4-[(1,3-benzodioxol-5-ylmethyl)amiono]-6-chloro-2-quinozolinyl]-4-piperidine-carboxylic acid, monosodium salt; (+)-cis-5,6a,7,9,9,9a-hexahydro-2-[4-(trifluoromethyl)-phenylmethyl-5-methyl-cyclopent-4,5]imidazo[2,1-b]purin-4(3H)one; furazlocillin; cis-2-hexyl-5-methyl-3,4,5,6a,7,8,9,9a-octahydrocyclopent[4,5]-imidazo[2,1-b]purin-4-one; 3-acetyl-1-(2-chlorobenzyl)-2-propylindole-6-carboxylate; 3-acetyl-1-(2-chlorobenzyl)-2-propylindole-6-carboxylate; 4-bromo-5-(3-pyridylmethylamino)-6-(3-(4-chlorophenyl)propoxy)-3-(2H)pyridazinone; I-methyl-5(5-morpholinoacetyl-2-n-propoxyphenyl)-3-n-propyl-1,6-dihydro-7H-pyrazolo(4,3-d)pyrimidin-7-one; 1-[4-[(1,3-benzodioxol-5-ylmethyl)arnino]-6-chloro-2-quinazolinyl]-4-piperidinecarboxylic acid, monosodium salt; Pharmaprojects No. 4516 (Glaxo Wellcome); Pharmaprojects No. 5051 (Bayer); Pharmaprojects No. 5064 (Kyowa Hakko; see WO 96/26940); Pharmaprojects No. 5069 (Schering Plough); GF-196960 (Glaxo Wellcome); E-8010 and E-4010 (Eisai); Bay-38-3045 & 38-9456 (Bayer) and Sch-51866.

The contents of the published patent applications and journal articles and in particular the general formulae of the therapeutically active compounds of the claims and exemplified compounds therein are incorporated herein in their entirety by reference thereto.

More preferred PDE5 inhibitors for use with the invention are selected from the group:
5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil);
(6R, 12aR)-2,3,6,7,12,12a-hexahydro-2-methyl-6-(3,4-methylenedioxyphenyl)-pyrazino[2′,1′:6,1]pyrido[3,4-b]indole-1,4-dione (IC-351);
2-[2-ethoxy-5-(4-ethyl-piperazin-1-yl-1-sulphonyl)-phenyl]-5-methyl-7-propyl-3H-imidazo[5, 1-f][1,2,4]triazin-4-one (vardenafil); and
5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one or 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one and pharmaceutically acceptable salts thereof.

A particularly preferred PDE5 inhibitor is 5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil) (also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methylpiperazine) and pharmaceutically acceptable salts thereof. Sildenafil citrate is a preferred salt.

Preferred agents for coadministration with the compounds of the present invention are PDE5 inhibitors, selective serotonin reuptake inhibitors (SSRIs), vasopressin V_{1A} antagonists, α-adrenergic receptor antagonists, NEP inhibitors, dopamine agonists and melanocortin receptor agonists as described above. Particularly preferred agents for coadministration are PDE5 inhibitors, SSRIs, and V_{1A} antagonists as described herein.

A suitable assay for determining the oxytocin antagonist activity of a compound is detailed herein below.

Oxytocin Receptor Beta-Lactamase Assay

Materials:

Cell Culture/Reagents

| A: cell culture | B: reagents |
|---|---|
| Nutrient Mixture | Oxytocin |
| F12 Ham's | OT receptor-specific antagonist |
| Foetal Bovine Serum (FBS) | Molecular grade Dimethyl Sulphoxide |
| | (DMSO) |
| Geneticin | Trypan Blue Solution 0.4% |
| Zeocin | CCF4-AM (Solution A) |
| Trypsin/EDTA | Pluronic F127s (Solution B) |
| PBS (phosphate buffered saline) | 24% PEG, 18% TR40 (Solution C) |
| HEPES | Probenecid (Dissolved at 200 mM |
| | in 200 mM NaOH, Solution D) |

Methods:
Cell Culture

Cells used are CHO-OTR/NFAT-β-Lactamase. The NFAT-β-lactamase expression construct was transfected into the CHO-OTR cell line and clonal populations were isolated via fluorescence activated cell sorting (FACS). An appropriate clone was selected to develop the assay.

| Growth Medium | Assay media |
|---|---|
| 90% F12 Nutrient Mix, 15 mM | 99.5% F12 Nutrient Mix, 15 mM |
| HEPES | HEPES |
| 10% FBS | 0.5% FBS |
| 400 μg/ml Geneticin | |
| 200 μg/ml Zeocin | |
| 2 mM L-Glutamine | |

Recovery of cells—A vial of frozen cells is thawed rapidly in 37° C. water bath and the cell suspension transferred into a T225 flask with 50ml of fresh growth medium and then incubated at 37° C., 5% CO₂ in an incubator until the cells adhered to the flask Replace media with 50ml of fresh growth media the following day. Culturing cells—CHO-OTR-NFAT-βLactamase cells were grown in growth medium. Cells were harvested when they reached 80-90% confluence removing the medium and washing with pre-warmed PBS. PBS was then removed and Trypsin/EDTA added (3 mls for T225 cm² flask) before incubating for 5 min in 37° C./5%CO₂ incubator. When cells were detached, pre-warmed growth media was added (7 mls for T225 cm² flask) and the cells re-suspended and mixed gently by pipetting to achieve single cell suspension. The cells were split into T225 flask at 1:10 (for 3 days growth) and 1:30 (for 5 days growth) ratio in 35 ml growth medium.

β-Lactamase Assay Method

Day 1

Cell Plate Preparation:

Cells grown at 80-90% confluence were harvested and counted. Suspensions of cells at 2×10⁵ cells/ml in growth medium were prepared and 30 μl of cells suspension added in 384-well, black clear-bottom plates. A blank plate containing diluents from each reagent was used for background subtraction. Plates were incubated at 37° C., 5% CO₂ overnight.

Day 2

Cells Stimulation:
10 μl antagonist/compound (diluted in assay media containing 1.25% DMSO=antagonist diluent) was added to appropriate wells and incubated for 15 minutes at 37° C., 5% CO₂
10 μl oxytocin, made up in assay media, was added to all wells and incubated for 4 hours at 37° C., 5% CO₂.
A separate 384-well cell plate was used to generate an oxytocin dose response curve. (10 μl antagonist diluent was added to every well. 10 μl of oxytocin was then added. The cells are then treated as per antagonist/compound cell plates).

Preparation of 1 ml of 6× Loading Buffer with Enhanced Loading Protocol (this requires scale-up according to number of plates to be screened)
12 μl of solution A (1 mM CCF4-AM in Dry DMSO) was added to 60 μl of solution B (100 mg/ml Pluronic-F127 in DMSO+0.1% Acetic Acid) and vortexed.
The resulting solution was added to 925 μl of solution C (24% w/w PEG400, 18% TR40 v/v in water).
75 μl of solution D was added (200 mM probenecid in 200 mM NaOH).
10 μl of 6× Loading Buffer was added to all wells and incubated for 1.5 hrs-2 hrs at room temperature in the dark.
The plates were read using an LJL Analyst, Excitation 405 nm, Emission 450 nm and 530 nm, gain optimal, lagtime 0.40 μs integration, 4 flashes, bottom reading.

Using the assay described above, the compounds of the present invention all exhibit oxytocin antagonist activity, expressed as a Ki value, of less than 500 nM. Preferred examples have Ki values of less than 200 nM and particularly preferred examples have Ki values of less than 50 nM. The compound of example 8 has a Ki value of 3 nM.

The invention is illustrated by the following non-limiting examples in which the following abbreviations and definitions are used:
Arbocel® Filtration agent, from J. Rettenmaier & Sohne, Germany
APCI+ Atmospheric Pressure Chemical Ionisation (positive scan)
CDCl₃ Chloroform-d1
d Doublet
dd Doublet of doublets
DMSO Dimethylsulfoxide
ES+ Electrospray ionisation positive scan.
eq Equivalent
¹H NMR Proton Nuclear Magnetic Resonance Spectroscopy
MS (Low Resolution) Mass Spectroscopy
m Multiplet
m/z Mass spectrum peak
q Quartet
s Singlet
t Triplet
δ Chemical shift

### Preparation 1

### Methyl 5-methoxy-2-nitrobenzoate

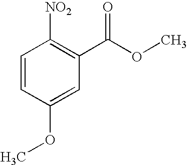

Fuming hydrochloric acid was passed through an ice-cooled solution of 5-methoxy-2-nitrobenzoic acid (10 g, 50.7 mmol) in methanol (70 mL) until saturated. The reaction mixture was warmed to room temperature for 18 hours and was then heated under reflux for 4 hours. The solvent was then evaporated under reduced pressure and the residue was partitioned between ethyl acetate and sodium hydrogen carbonate solution. The organic layer was separated and washed with sodium hydrogen carbonate solution and brine, dried over magnesium sulfate, and concentrated in vacuo to give the title compound as a brown oil in 77% yield, 8.23 g. ¹H NMR (CDCl₃, 400 MHz), δ: 3.90-3.95 (m, 6H), 6.98-7.05 (m, 2H), 8.00-8.05 (m, 1H). MS APCI+ m/z 212 [MH]⁺

### Preparation 2

### (5-Methoxy-2-nitrophenyl)methanol

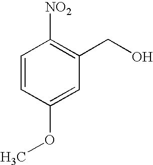

The product of preparation 1 (4 g, 18.9 mmol) was dissolved in tetrahydrofuran (30 mL) and stirred at room temperature. Sodium borohydride (1.43 g, 37.9 mmol) in methanol (5 mL) was added dropwise and the mixture was heated under reflux. After 90 minutes, the reaction was treated with additional sodium borohydride (100 mg) in methanol (1 mL) and heating continued at reflux for a further 90 minutes. The reaction was then quenched with water (10 mL) and 2M sodium hydroxide (10 mL) and the tetrahydrofuran was evaporated under reduced pressure to leave an aqueous residue. The residue was extracted with dichloromethane (3×5 mL) and the combined organic fractions were dried over magnesium sulfate and concentrated in vacuo to give a light brown solid. Trituration of the solid with diethyl ether afforded the title compound as a white solid in 78% yield, 2.69 g.

¹H NMR (CDCl₃, 400 MHz), δ: 3.82 (s, 3H), 4.95 (s, 2H), 6.98-7.05 (m, 1H), 7.30 (s, 1H), 8.07 (d, 1H).

### Preparation 3

### (2-Amino-5-methoxyphenyl)methanol

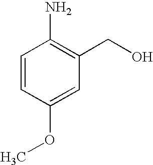

The nitro product of preparation 2 (620 mg, 3.38 mmol) and 10% Pd/C (60 mg) was added to a mixture of ethanol and water (9:1 by volume, 20 mL) and the mixture was stirred under 414 kPa (60 psi) of hydrogen gas for 1 hour. The reaction mixture was then filtered through Arbocel® and the filtrate was evaporated under reduced pressure. The resulting oily residue was dissolved in dichloromethane, dried over magnesium sulfate, and concentrated in vacuo to give a solid. Trituration of the solid with diethyl ether afforded the title product as a white solid in 42% yield, 220 mg.

¹H NMR (CDCl₃, 400 MHz), δ: 3.78 (s, 3H), 4.62 (s, 2H), 6.60-6.75 (m, 3H). MS APCI+ m/z 154 [MH]⁺

### Preparation 4

### 4-Bromo-N′-(chloroacetyl)benzohydrazide

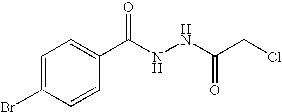

N-methylmorpholine (173 mmol, 17.6 g) was added to an ice-cooled suspension of 4-bromobenzhydrazide (25 g, 116 mmol) in dichloromethane (350 mL). Chloroacetylchloride (11.1 mL, 139 mmol) was added drop-wise and the mixture was stirred for 90 minutes at 25° C. The reaction slurry was then filtered and the collected solid was stirred in water (200 mL). The precipitate was then filtered off, washing through with methanol and diethyl ether, and dried to afford the title compound as a solid in 76% yield, 25.7 g.

¹H NMR (DMSO-D₆, 400 MHz), δ: 4.10 (s, 2H), 7.70 (d, 2H), 7.80 (d, 2H), 10.40 (s, 1H), 10.6 (d, 1H). MS APCI+ m/z 291 [MH]⁺

### Preparation 5

### 2-(4-Bromophenyl)-5-(chloromethyl)-1,3,4-oxadiazole

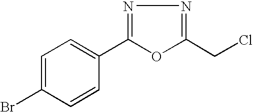

The product of preparation 4 (25.5 g, 87 mmol) was added to phosphorous oxychloride (90 mL) and the reaction mixture heated to 110° C. for 3 hours. The mixture was then allowed to cool to room temperature and was concentrated in vacuo. The residue was diluted with water (500 mL) and basified with saturated sodium hydrogen carbonate solution. The aqueous mixture was extracted with ethyl acetate (2×250 mL) and the organic phases were combined. These were washed with water (500 mL) and brine (250 mL), dried over sodium sulfate and concentrated in vacuo to afford the title product in 77% yield, 18.2 g.

¹H NMR (CDCl₃, 400 MHz), δ: 4.80 (s, 2H), 7.70 (d, 2H), 8.00 (d, 2H). MS APCI+ m/z 275 [MH]⁺

### Preparation 6

### 2-({[5-(4-Bromophenyl)-1,3,4-oxadiazol-2-yl]methoxy}methyl)-4-methoxyaniline

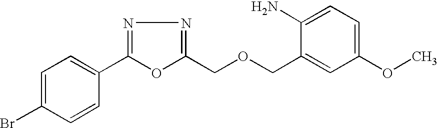

The product of preparation 3 (300 mg, 2.0 mmol) in tetrahydrofuran (3 mL) was added dropwise to a suspension of sodium hydride (60% dispersion in mineral oil, 54 mg, 2.3 mmol) in tetrahydrofuran (2 mL). The mixture was stirred for 45 minutes and the temperature was maintained at −10° C. The product of preparation 5 (357 mg, 1.3 mmol) in tetrahydrofuran (3 mL) was added dropwise and the mixture was stirred for a further hour. The temperature was then allowed to increase to 25° C. and the reaction was quenched with sodium hydrogen carbonate solution (5 mL), and diluted with ethyl acetate (20 mL) and water (10 mL). The organic phase was separated, washed with brine (10 mL), dried over magnesium sulfate and concentrated in vacuo to give an oily residue. Purification of the oil by column chromatography on silica gel, eluting with dichloromethane:methanol, 100:0 to 97:3, afforded the title compound in 59% yield, 300 mg. MS APCI+ m/z 390, 392 [MH]⁺

### Preparation 7

### 1-(4-Bromophenyl)-8-methoxy-4H,6H-[1,2,4]triazolo[4,3-a][4,1]benzoxazepine

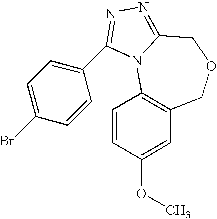

The product of preparation 6 (300 mg) and para-toluenesulfonic acid (cat) were suspended in toluene (5 mL) and heated to 80° C. for 5 hours. The reaction mixture was then cooled and was loaded onto a silica gel column. Elution with pentane:ethyl acetate, 90:10 to 0:100, afforded the title compound in 45% yield, 130 mg. ¹H NMR (CDCl₃, 400 MHz), δ: 3.86 (s, 3H), 4.60 (s, 2H), 4.70 (s, 2H), 6.80-6.95 (m, 2H), 7.05 (brs, 1H), 7.40-7.60 (m, 4H). MS APCI+ m/z 372, 374 [MH]⁺

### Preparation 8

### 2-(Dibromomethyl)-6-methoxy-3-nitropyridine

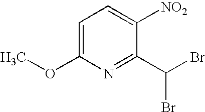

A solution of potassium tert-butoxide (29 g, 260 mmol) in tetrahydrofuran (100 mL) and N,N-dimethylformamide (30 mL) was cooled to −70° C. and a mixture of 2-methoxy-5-nitropyridine (10 g, 64.9 mmol) and bromoform (6.5 mL, 74.6 mmol) dissolved in tetrahydrofuran (30 mL) and N,N-dimethylformamide (15 mL), was added dropwise over a period of 2 hours. Acetic acid (30 mL) was then added slowly and the reaction was allowed to warm to 25° C. The reaction mixture was poured into ice water and was extracted with ethyl acetate (4×100 mL). The combined organic extracts were washed with 2M hydrochloric acid (3×20 mL), sodium hydrogen carbonate solution (50 mL) and brine (50 mL), dried over magnesium sulfate and concentrated in vacuo. The residue was dissolved in methanol and was heated under reflux for 5 minutes in the presence of charcoal. The suspension was filtered and the filtrate was concentrated in vacuo. Trituration of the residue with pentane afforded the title product as a pale brown solid in 62% yield, 13.18 g.

¹H NMR (CDCl₃, 400 MHz), δ: 4.20 (s, 3H), 6.80-6.90 (d, 1H), 7.63 (s, 1H), 8.20-8.30 (d, 1H).

### Preparation 9

### 6-Methoxy-3-nitropyridine-2-carbaldehyde

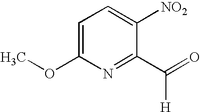

A solution of silver nitrate (36.5 g, 214.7 mmol) in water (50 mL) was added to a suspension of the product of preparation 8 (28 g, 85.9 mmol) in ethanol (200 mL), and the mixture was heated under reflux for 5 hours. The cooled reaction mixture was then evaporated under reduced pressure and the residue was suspended in ethyl acetate (100 mL). The resulting precipitate was removed by filtration, washing through with water and ethyl acetate, and the layers of the filtrate were separated. The aqueous layer was extracted with dichloromethane (×3) and re-filtered; the organic layer was washed with water (50 mL), 2M hydrochloric acid (50 mL) and brine (50 mL). The combined organic fractions were dried over magnesium sulfate and concentrated in vacuo to give an oily residue that crystallised upon scratching. The crystallised solid was then triturated diethyl ether and was filtered. The filtrate was concentrated in vacuo and the residue was purified by column chromatography on silica gel, eluting with pentane:ethyl acetate, 85:15 to 75:25, to afford the title compound in 13% yield, 2 g. ¹H NMR (CDCl₃, 400 MHz), δ: 4.10 (s, 3H), 6.98 (d, 1H), 8.25 (d, 1H), 10.30 (s, 1H). MS APCI+ m/z 197 [MH]⁺

### Preparation 10

### (6-Methoxy-3-nitropyridin-2-yl)methanol

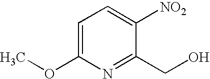

Sodium borohydride (685 mg, 18.1 mmol) was added to an ice-cold solution of the product of preparation 9 (3 g, 16.5 mmol) in ethanol (30 mL), and the mixture was stirred for 2 hours at room temperature. The reaction was then quenched by the addition of sodium hydrogen carbonate solution (25 mL) and the solvent was evaporated under reduced pressure. The aqueous residue was diluted with dichloromethane (75 mL) and water (25 mL), the phases were separated and the aqueous phase was extracted with dichloromethane (2×30 mL). The combined organic solutions were washed with 2M hydrochloric acid (30 mL), sodium hydrogen carbonate solution and brine, dried over magnesium sulfate and concentrated in vacuo to give an orange solid. The solid was then dissolved in methanol (25 mL) and decolourised by charcoal and triturated with pentane and diethyl ether to afford the title compound as a pale brown solid in 46% yield, 1.4 g. ¹H NMR (CDCl₃, 400 MHz), δ: 4.10 (s, 3H), 4.20 (brs, 1H), 5.15 (s, 2H), 6.80 (d, 1H), 8.45 (d, 1H). MS APCI+ m/z 185 [MH]⁺

### Preparation 11

### (3-Amino-6-methoxypyridin-2-yl)methanol

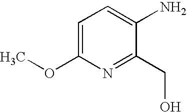

The nitro product of preparation 10 (1.4 g, 7.6 mmol) and 10% Pd/C (100 mg) were added to a mixture of ethanol and water (9:1 by volume, 40 mL) and the mixture was stirred under 414 kPa (60 psi) of hydrogen gas for 1 hour. The reaction mixture was then filtered through Arbocel® and the filtrate was concentrated in vacuo. Trituration of the residue with dichloromethane gave the title product as a pale orange solid in quantitative yield. ¹H NMR (CDCl₃, 400 MHz), δ: 3.90 (s, 3H), 4.60 (s, 2H), 5.15 (s, 2H), 6.60 (d, 1H), 7.05 (d, 1H). MS APCI+ m/z 155 [MH]⁺. Micro analysis found (%); C (54.33), H (6.56), N (17.82); C₇H₁₀N₂O₂ requires (%); C (54.53), H (6.54), N (18.17).

### Preparation 12

### 2-Chloro-N-[2-(hydroxymethyl)-6-methoxypyridin-3-yl]acetamide

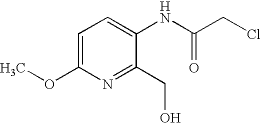

A solution of chloroacetyl chloride (977 mg, 8.65 mmol) in dichloromethane (2 mL) was added dropwise to an ice-cooled solution of the product of preparation 11 (1.16 g, 7.52 mmol) and N-ethyldiisopropylamine (1.16 g, 9.03 mmol) in dichloromethane (8 mL). The mixture was allowed to warm to 25° C. and was stirred for 1 hour. Additional chloroacetyl chloride (few drops) and N-ethyldiisopropylamine (few drops) were added and stirring continued for a further 90 minutes. The reaction mixture was then diluted with water (10 mL) and was washed with sodium hydrogen carbonate solution (5 mL) and brine (5 mL). The organic phase was separated, dried over magnesium sulfate and concentrated in vacuo to afford the title compound as a brown oil in 96% yield, 1.67 g. ¹H NMR (CDCl₃, 400 MHz), δ: 3.95 (s, 3H), 4.20 (s, 2H), 4.78 (s, 2H), 6.70 (d,1H), 8.05 (d, 1H), 8.75-8.90 (brs, 1H). MS APCI+ m/z 231, 233 [MH]⁺

### Preparation 13

### 7-Methoxy-1,5-dihydropyrido[3,2-e][1,4]oxazepin-2(3H)-one

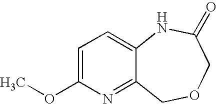

Potassium tert-butoxide (113 mg, 1.01 mmol) was added to a solution of the product of preparation 12 (106 mg, 0.46 mmol) in tert-butanol (1 mL) and the mixture was stirred for 15 minutes at room temperature. The solvent was then evaporated under reduced pressure and the residue was partitioned between diethyl ether and water. The precipitate was filtered off, washing through with diethyl ether, and the phases were separated. The aqueous layer was extracted with dichloromethane (×3) and the combined organic layers were dried over sodium sulfate and concentrated in vacuo to give the to afford the title compound as a white solid in 56% yield, 50 mg. ¹H NMR (DMSO-D₆, 400 MHz), δ: 3.78 (s, 3H), 4.40 (s, 2H), 4.75 (s, 2H), 6.70 (d, 1H), 7.45 (d, 1H), 10.00 (brs, 1H). MS APCI+ m/z 195 [MH]⁺. Micro analysis found (%); C (55.52), H (5.14), N (14.40); C₉H₁₀N₂O₃ requires (%); C (55.66), H (5.19), N (14.43).

### Preparation 14

### 7-Methoxy-1,5-dihydropyrido[3,2-e][1,4]oxazepine-2(3H)-thione

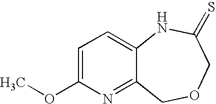

Sodium carbonate (470 mg, 4.42 mmol) was added to tetrahydrofuran (10 mL) and the suspension was stirred at 0° C. Phosphorous pentasulfide (2 g, 4.42 mmol) and the product of preparation 13 (780 mg, 4.01 mmol) were then added and the mixture was stirred for 3 hours at 25° C. The reaction mixture was partitioned between dichloromethane (50 mL) and 0.88 ammonium hydroxide solution (20 mL), and the aqueous phase was separated and re-extracted with dichloromethane (2×20 mL). The combined organic phase was washed with 2M hydrochloric acid (2×10 mL), sodium hydrogen carbonate solution and brine, dried over sodium sulfate and concentrated in vacuo to afford the title compound in 31% yield, 260 mg.

¹H NMR (DMSO-D₆, 400 MHz), δ: 3.80(s, 3 H), 4.68 (s, 2H), 4.82 (s, 2H), 6.70 (d, 1H), 7.80 (d, 1H), 11.90-12.00 (brs, 1H). MS APCI+ m/z 211 [MH]⁺

### Preparation 15

### 5-Bromopyridine-2-carbohydrazide

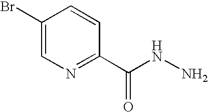

Hydrazine hydrate (5.3 mL, 108.3 mmol) was added to an ice-cooled solution of the product of preparation methyl 5-bromopyridine-2-carboxylate [(7.80 g, 36.1 mmol) J. Org. Chem, 66, 605-608] dissolved in methanol (100 mL) and the mixture was heated under reflux for 1 hour. The solvent was concentrated to low volume under reduced pressure and was then cooled. The precipitate that formed was filtered off, washed with methanol and dried in vacuo to give the title product in 89% yield, 6.94 g.

¹H NMR (DMSO-D₆, 400 MHz), δ: 4.40-4.65 (brs, 2H), 7.90-7.95 (d, 1H), 8.21 (d, 1H), 8.70 (s, 1H), 9.90-10.0 (brs, 1H). MS APCI+ m/z 216, 218 [MH]⁺

### Preparation 16

### 1-(5-Bromopyridin-2-yl)-8-methoxy-4H,6H-pyrido[3,2-e][1,2,4]triazolo[3,4-c][1,4]oxazepine

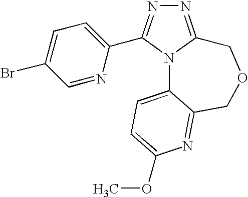

The products of preparation 14 (266 mg, 1.26 mmol) and preparation 15 (300 mg, 1.39 mmol) were dissolved in n-butanol (5 mL) and the mixture was heated at 110° C. for 10 hours. The reaction mixture was then cooled to 4° C. for 30 minutes and the resulting precipitate was filtered off, washed with diethyl ether and dried. Purification of this solid by column chromatography on silica gel, eluting with dichloromethane:methanol, 100:0 to 98:2 afforded the title compound as a solid in 64% yield, 300 mg.

¹H NMR (CDCl₃, 400 MHz), δ: 3.95 (s, 3H), 4.60 (s, 2H), 4.68 (s, 2H), 6.70 (d, 1H), 7.47 (d, 1H), 7.90-7.95 (d, 1H), 8.13 (d, 1H), 8.42 (s, 1H). MS APCI+ m/z 374, 376 [MH]⁺

### Preparation 17

### Benzyl{[5-(4-bromophenyl)-1,3,4-oxadiazol-2-yl]methyl}amine

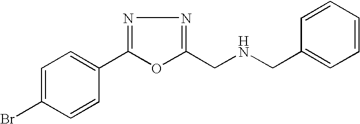

Potassium carbonate (5.04 g, 36.55 mmol) was added to an ice-cooled solution of the product of preparation 5 (5 g, 18.28 mmol) in acetonitrile (50 mL). Benzylamine (2.25 g, 21.02 mmol) in acetonitrile (5 mL) was added dropwise and the resulting mixture was stirred at room temperature for 24 hours and at 50° C. for 6 hours. The reaction mixture was then filtered and the filtrate was evaporated under reduced pressure. The residue was dissolved in ethyl acetate (100 mL), washed with water (2×50 mL) and brine (50 mL), dried over magnesium sulfate and concentrated in vacuo. Trituration of the residue with diethyl ether afforded the title compound as a white solid in 52% yield, 3.3 g.

¹H NMR (CDCl₃, 400 MHz), δ: 2.30-2.70 (brs, 1H), 3.75 (s, 2H), 3.92 (s, 2H), 7.05-7.20 (m, 5H), 7.47 (d, 2H), 7.75 (d, 2H). MS APCI+ m/z 344, 346 [MH]⁺

### Preparation 18

### Benzyl{[5-(4-bromophenyl)-1,3,4-oxadiazol-2-yl]methyl}[(6-methoxy-3-nitropyridin-2-yl)methyl]amine

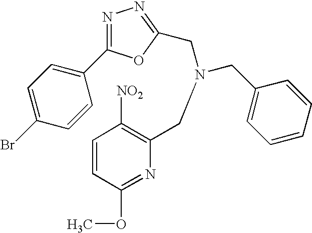

Sodium triacetoxyborohydride (2.64 g, 12.46 mmol) was added slowly to an ice-cooled solution of the products of preparations 17 (3.3 g, 9.59 mmol) and 9 (2.1 g, 11.5 mmol) in dichloromethane (30 mL) and the mixture was allowed to stir for 6 hours at 25° C. The reaction mixture was then diluted with dichloromethane (10 mL), washed with sodium hydrogen carbonate solution (2×5mL), brine (5 mL), dried over magnesium sulfate and concentrated in vacuo. Purification of the residue by column chromatography on silica gel, eluting with dichloromethane:methanol, 100:0 to 98:2 afforded the title compound in 75% yield, 4.4 g.

¹H NMR (CDCl₃, 400 MHz), δ: 3.85-4.0 (brs, 1H), 4.05 (s, 3H), 4.10 (s, 2H), 4.40 (s, 2H), 6.67 (d, 1H), 7.18-7.35 (m, 5H), 7.67 (d, 2H), 7.93 (d, 2H), 8.13 (d, 1H). MS APCI+ m/z 510, 512 [MH]⁺

### Preparation 19

### 5-Benzyl-1-(4-bromophenyl)-8-methoxy-5,6-dihydro-4H-pyrido[2,3-f][1,2,4]triazolo[4,3-a][1,4]diazepine

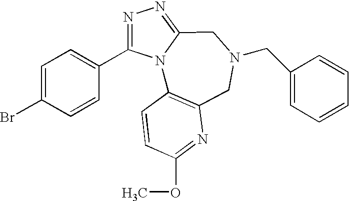

Iron powder (1.43 g, 25.57 mmol), was added to the product of preparation 18 (4.35 g, 8.52 mmol) suspended in acetic acid (45 mL) and the mixture was heated at 60° C. for 3 hours. The solvent was then evaporated under reduced pressure and the residue was dissolved in ethyl acetate, filtered and washed with citric acid, sodium hydrogen carbonate solution and brine. The organic layer was separated, dried over magnesium sulfate and concentrated in vacuo to afford the title compound as a pale brown solid in 61% yield, 2.4 g. ¹H NMR (CDCl₃, 400 MHz), δ: 3.60-4.10 (brm, 6H), 4.05 (s, 3H), 6.70 (d, 1H), 7.10 (d, 1H), 7.30-7.60 (m, 9H). MS APCI+ m/z 462, 464 [MH]⁺

### EXAMPLE 1

### 8-Methoxy-1-(2′-methoxybiphenyl-4-yl)-4H,6H-[1,2,4]triazolo[4,3-a][4,1]benzoxazepine

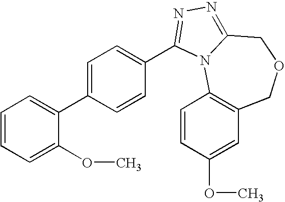

The product of preparation 7 (123 mg, 0.33 mmol) and 2-methoxybenzeneboronic acid (75 mg, 0.49 mmol) were dissolved in 1,2 dimethoxyethane (4 mL). Sodium carbonate (70 mg, 0.66 mmol) in water (1 mL) and tetrakis-(triphenylphosphine) palladium (20 mg, cat) were added and the mixture was heated under reflux for 90 minutes. The reaction mixture was then cooled and filtered and the filtrate was concentrated in vacuo. The aqueous residue was diluted with ethyl acetate and the mixture was washed with sodium hydrogen carbonate and brine. The organic layer was separated, dried over magnesium sulfate and concentrated in vacuo. Purification of the residue by column chromatography on silica gel, eluting with ethyl acetate:pentane, 20:80 to 100:0, afforded the title compound in 61% yield, 80 mg. ¹H NMR (CDCl₃, 400 MHz), δ: 3.80-3.86 (m, 6H), 4.60-4.70 (m, 4H), 6.90-7.60 (m,11H). MS APCI+ m/z 400 [MH]⁺

### EXAMPLES 2 and 3

The following compounds, of the general formula shown below, were prepared by a method similar to that described for example 1 using the appropriate starting material.

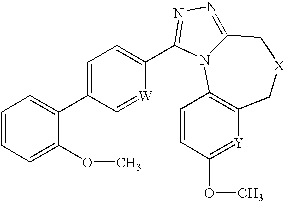

| Example | W | X | Y | Data | Yield |
|---|---|---|---|---|---|
| 2 | N | O | N | ¹H NMR(CDCl₃, 400MHz) δ: 3.82(s, 3H), 4.02(s, 3H), 4.70(s, | 51% |
| | | | | 2H), 4.80(s, 2H) 6.80(d, 1H), 7.00-7.10(m, 3H), 7.30-7.70(m, | |
| | | | | 3H), 8.05(d, 1H), 8.32(d, 1H), 8.62(s, 1H) | |
| | | | | MS APCl + m/z 386 [MH]⁺ | |
| 3 | CH | N—CH₂Ph | N | ¹H NMR(CDCl₃, 400MHz) δ: 3.82(s, 3H), 4.05(brm, 6H), | 92% |
| | | | | 4.06(s, 3H), 6.65-6.80(brd, 1H), 6.95-7.10(m, 2H) 7.20- | |
| | | | | 7.65(m, 12H). MS APCl + m/z 490 [MH]⁺. Micro analysis | |
| | | | | found (%); C(72.87), H(5.57), N(13.73); C₃₀N₂₇N₅O₂ 0.3 H₂O | |
| | | | | requires (%); C(72.80), H(5.62), N(14.15) | |

### EXAMPLE 4

### 8-Methoxy-1-(2′-methoxybiphenyl-4-yl)-5,6-dihydro-4H-pyrido[2,3-f][1,2,4]triazolo[4,3-a][1,4]diazepine

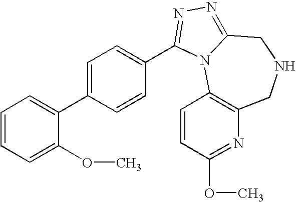

To a solution of the product of example 3 (869 mg, 1.77 mmol) in ethanol (20 mL) was added 20% Pd(OH)₂/C (20 mg) and ammonium formate (133 mg, 2.1 mmol) and the mixture was warmed at 80° C. The reaction was monitored by tic analysis and extra portions of 20% Pd(OH)₂/C (25 mg) and ammonium formate (500 mg) were added at hourly intervals until all of the starting material had been consumed. The reaction mixture was then heated under reflux for 20 minutes and filtered. The filtrate was evaporated under reduced pressure and the residue was partitioned between ethyl acetate (30 mL) and water (30 mL). The organic phase was separated and washed with sodium hydrogen carbonate solution (10 mL) and brine (10 mL), dried over magnesium sulfate and concentrated in vacuo. Trituration of the residue with diethyl ether afforded the title compound in 58% yield, 410 mg.

¹H NMR (CDCl₃, 400 MHz), δ: 2.20-2.60 (brs, 1H), 3.75 (s, 3H), 3.95 (s, 3H), 4.00 (brm, 4H), 6.63 (d, 1H), 6.90-7.00 (m, 2H), 7.15-7.20 (d, 1H), 7.22-7.30 (m, 2H), 7.45-7.55 (m, 4H). MS APCI+ m/z 400 [MH]⁺

### EXAMPLE 5

### 8-Methoxy-5-methyl-1-(2′-methoxybiphenyl-4-yl)-5,6-dihydro-4H-pyrido[2,3-f][1,2,4]triazolo[4,3-a][1,4]diazepine

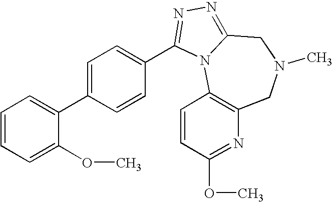

The product of example 4 (100 mg, 0.25 mmol) and formaldehyde (37% 81 μL, 1.0 mmol) were stirred in dichloromethane (3 mL) at room temperature for 25 minutes. Sodium triacetoxyborohydride (64 mg, 0.30 mmol) was added and the mixture was stirred for 1 hour. The reaction mixture was then treated with additional formaldehyde (37%, 40 μL, 0.5 mmol) and sodium triacetoxyborohydride (30 mg, 0.15 mmol) and stirring continued for a further hour. Sodium hydrogen carbonate solution (3 mL) was then added and the mixture was stirred for 10 minutes. The organic layer was then separated, washed with sodium hydrogen carbonate solution (3 mL) and brine (5 mL), dried over magnesium sulfate and concentrated in vacuo to afford the title compound as a white foam in 77% yield, 83 mg.

¹H NMR (CDCl₃, 400 MHz), δ: 2.60 (s, 3H), 3.70 (s, 3H), 3.75-3.95 (brm, 4H), 4.05 (s, 3H), 6.70 (d, 1H), 6.95-7.10 (m, 2H), 7.20-7.40 (m, 3H), 7.50-7.65 (m, 4H). MS APCI+ m/z 414 [MH]⁺. Micro analysis found (%); C (68.20), H (5.64), N (16.28); C₂₄H₂₃N₅O₂ 0.5 H₂O requires (%); C (68.23), H (5.73), N (16.58).

### EXAMPLE 6

### 5-Acetyl-8-methoxy-1-(2′-methoxybiphenyl-4-yl)-5,6-dihydro-4H-pyrido[2,3-f][1,2,4]triazolo[4,3-a][1,4]diazepine

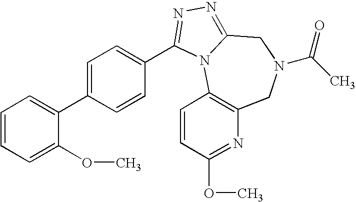

Acetyl chloride (23 mg, 0.29 mmol) was added to an ice-cooled solution of the product of example 4 (90 mg, 0.22 mmol) and N-ethyldiisopropylamine (54 μL, 0.31 mmol) in dichloromethane (3 mL). The resulting mixture was allowed to warm to room temperature and was stirred for 1 hour. Dichloromethane (3 mL) was added and the reaction mixture was washed with water (3 mL), 2M hydrochloric acid (3 mL), sodium hydrogen carbonate solution (3 mL) and brine. The organic phase was dried over magnesium sulfate and concentrated in vacuo. Trituration of the residue with diethyl ether afforded the title compound as a solid in 74% yield, 60 mg. ¹H NMR (CDCl₃, 400 MHz), δ: 2.30-2.40 (m, 3H), 3.80 (s, 3H), 4.00 (s, 3H), 4.60-5.00 (m, 4H), 6.70-6.80 (m, 1H), 6.98-7.10 (m, 2H), 7.25-7.40 (m, 3H), 7.50-7.70 (m, 4H). MS APCI+ m/z 442 [MH]⁺. Micro analysis found (%); C (67.31), H (5.32), N (15.47); C₂₅H₂₃N₅O₂ 0.3 H₂O requires (%); C (67.19), H (5.32), N (15.67).

### EXAMPLES 7 and 8

The following compounds, of the general formula shown below, were prepared by a method similar to that described for example 6 using the product of example 4 and the appropriate chloride.

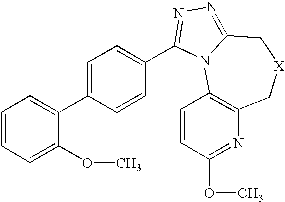

| Example | X | Data | Yield |
|---|---|---|---|
| 7 | N—SO₂CH₃ | ¹H NMR(CDCl₃, 400MHz) δ: 2.96(s, 3H), 3.80(s, 3H), 4.02(s, 3H), | 70% |
| | | 4.40-4.80(m, 4H) 6.80(d, 1H), 6.97-7.10(m, 2H), 7.25-7.40(m, 3H) | |
| | | 7.50-7.65(m, 4H). MS APCl + m/z 478 [MH]⁺ | |
| | | Micro analysis found (%); C(59.99), H(4.98), N(14.35); | |
| | | C₂₄H₂₃N₅O₄S requires (%); C(60.36), H(4.85), N(14.66) | |
| 8 | N—C(O)OCH₃ | ¹H NMR(CDCl₃, 400MHz) δ: 3.82(m, 6H), 4.02(s, 3H), 4.60- | 73% |
| | | 4.90(m, 4H), 6.73(d, 1H), 6.95-7.08(m, 2H), 7.20-7.40(m, 3H), | |
| | | 7.50-7.63(m, 4H). MS APCl + m/z 458 [MH]⁺ | |
| | | Micro analysis found (%); C(65.30), H(5.07), N(15.11); C₂₅H₂₃N₅O₄ | |
| | | 0.4H₂O requires (%); C(66.92), H(5.35), N(15.61) | |

### EXAMPLE 9

### 8-Methoxy-1-(2′-methylbiphenyl-4-yl)-4H,6H-[1,2,4]triazolo[4,3-a][4,1]benzoxazepine

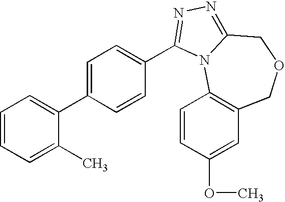

The title compound was prepared from the product of preparation 7 and (2-methylphenyl)boronic acid, using a similar method to the preparation of example 1, in 29% yield, 41 mg.

¹H NMR (CDCl₃, 400 MHz), δ: 2.25 (s, 3H), 3.90 (s, 3H), 4.62 (s, 2H), 4.75 (s, 2H), 6.90-7.65 (m, 11H). MS APCI+ m/z 400 [MH]⁺. Micro analysis found (%); C (72.98), H (5.45), N (10.14); C₂₄H₂₁N₃O₂ 0.75 H₂O requires (%); C (72.62), H (5.71), N (10.59).

### EXAMPLES 10 and 11

The following compounds, of the general formula shown below, were prepared by a method similar to that described for example 9 using the appropriate starting material.

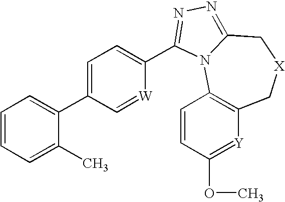

| Example | W | X | Y | Data | Yield |
|---|---|---|---|---|---|
| 10 | N | O | N | ¹H NMR(CDCl₃, 400MHz) δ: 2.30(s, 3H), 4.02(s, 3H), 4.70(s, | 76% |
| | | | | 2H), 4.80(s, 2H) 6.80(d, 1H), 7.20-7.65(m, 6H), 8.35-8.40(d, | |
| | | | | 1H), 8.42(s, 1H). MS APCl + m/z 386 [MH]⁺ | |
| 11 | CH | N—CH₂Ph | N | ¹H NMR(CDCl₃, 400MHz) δ: 2.30(s, 3H), 3.75-4.05(brm, | 98% |
| | | | | 6H), 4.06(s, 3H), 6.74(d, 1H), 7.20-7.60(m, 14H) | |
| | | | | MS APCl + m/z 474 [MH]⁺ | |

### EXAMPLE 12

### 8-Methoxy-1-(2′-methylbiphenyl-4-yl)-5,6-dihydro-4H-pyrido[2,3-f][1,2,4]triazolo[4,3-a][1,4]diazepine

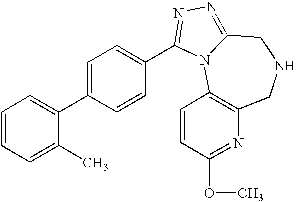

The title compound was prepared from the product of example 11, using a method similar to that described in example 4. However, this compound was purified by column chromatography on silica gel, eluting with dichloromethane: methanol, 100:0 to 95:5, to afford the title product as a white foam in 65% yield, 371 mg. ¹H NMR (CDCl₃, 400 MHz), δ: 2.20-2.60 (brm, 4H), 4.05 (s, 3H), 4.10-4.25 (m, 4H), 6.70 (d, 1H), 7.15-7.30 (m, 5H), 7.37 (d, 2H), 7.57 (d, 2H). MS APCI+ m/z 384 [MH]⁺. Micro analysis found (%); C (69.06), H (5.52), N (17.22); C₂₈H₂₁N₅O 0.25 DCM requires (%); C (69.01), H (5.36), N (17.31).

### EXAMPLE 13

### 8-Methoxy-5-methyl-1-(2′-methylbiphenyl-4-yl)-5,6-dihydro-4H-pyrido[2, 3-f][1,2,4]triazolo[4,3-a][1,4]diazepine

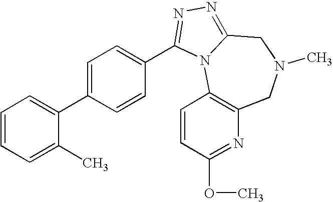

The product of example 12 (90 mg, 0.23 mmol) and formaldehyde (37%, 76 μL, 0.94 mmol) were stirred in dichloromethane (3 mL) at room temperature for 25 minutes. Sodium triacetoxyborohydride (60 mg, 0.28 mmol) was added and stirring continued for a further hour. Sodium hydrogen carbonate solution (3 mL) was then added and the mixture was stirred for 10 minutes. The organic layer was then separated, washed with sodium hydrogen carbonate solution (3 mL) and brine (5 mL), dried over magnesium sulfate and concentrated in vacuo to afford the title compound as a white foam in 90% yield, 83 mg.

¹H NMR (CDCl₃, 400 MHz), δ: 2.25 (s, 3H), 2.65-2.75 (brs, 3H), 3.70-4.20 (brm, 4H), 4.05 (s, 3H), 6.75 (d, 1H), 7.20-7.35 (m, 5H), 7.38 (d, 2H), 7.60 (d, 2H). MS APCI+ m/z 398 [MH]⁺. Micro analysis found (%); C (70.64), H (5.88), N (17.08); C₂₄H₂₃N₅O 0.5 H₂O requires (%); C (70.92), H (5.95), N (17.23).

### EXAMPLE 14

### 5-Acetyl-8-methoxy-1-(2′-methylbiphenyl-4-yl)-5,6-dihydro-4H-pyrido[2,3-f][1,2,4]triazolo[4,3-a][1,4]diazepine

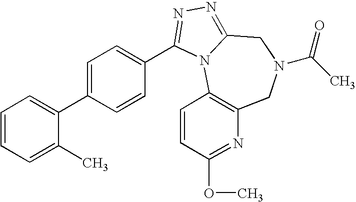

The title compound was prepared from the product of example 12, using a method similar to that described in example 6, as a solid in 67% yield, 60 mg.

¹H NMR (CDCl₃, 400 MHz), δ: 2.30 (s, 3H), 2.65-2.75 (m, 3H), 4.00 (s, 3H), 4.65-5.0 (brm, 4H), 6.70-6.80 (m, 1H), 7.20-7.65 (m, 9H). MS APCI+ m/z 426 [MH]⁺. Micro analysis found (%); C (69.04), H (5.51), N (15.99); C₂₅H₂₃N₅O₂O 0.5 H₂O requires (%); C (69.11), H (5.57), N (16.12).

### EXAMPLES 15 and 16

The following compounds, of the general formula shown below, were prepared by a method similar to that described for example 14 using the product of example 12 and the appropriate chloride.

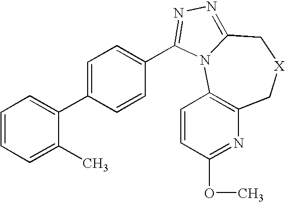

| Example | X | Data | Yield |
|---|---|---|---|
| 15 | N—SO₂CH₃ | ¹H NMR(CDCl₃, 400MHz) δ: 2.25(s, 3H), 2.98(s, 3H), 4.02(s, 3H), | 59% |
| | | 4.40-4.80(m, 4H) 6.80(d, 1H), 7.20-7.65(m, 5H), 7.41(d, 2H), | |
| | | 7.58(d, 2H). MS APCl + m/z 462 [MH]⁺ | |
| 16 | N—C(O)OCH₃ | ¹H NMR(CDCl₃, 400MHz) δ: 2.26(s, 3H), 3.80(s, 3H), 4.02(s, 3H), | 58% |
| | | 4.60-4.85(m, 4H), 6.75(d, 1H), 7.20-7.34(m, 5H), 7.39(d, 2H), | |
| | | 7.57(d, 2H). MS APCl + m/z 442 [MH]⁺ | |
| | | Micro analysis found (%); C(67.04), H(5.26), N(15.52); C₂₅H₂₃N₅O₃ | |
| | | 0.4H₂O requires (%); C(66.92), H(5.35), N(15.61) | |

### EXAMPLES 17 to 26

The following compounds, of the general formula shown below, were prepared by the method that described for example 1 using the compound of preparation 19 and the appropriate commercially available aryl boronic acid or ester starting material.

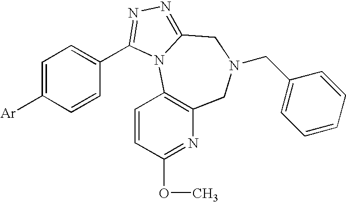

| | | Data: MS |
|---|---|---|
| | | APCl + m/z |
| Ex. | Ar | [MH]⁺ |
| 17 | 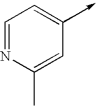 | 475 |
| 18 | 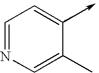 | 475 |
| 19 | 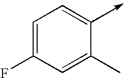 | 492 |
| 20 | 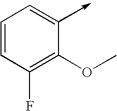 | 508 |
| 21 | 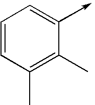 | 488 |
| 22 | 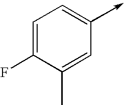 | 492 |
| 23 | 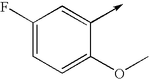 | 508 |
| 24 | 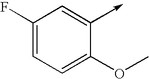 | 508 |
| 25 | 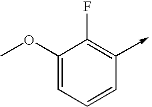 | 508 |
| 26 | 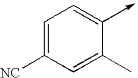 | 499 |

### EXAMPLE 27

### 8-Methoxy-5-methyl-1-(2′-methylbiphenyl-4-yl)-5,6-dihydro-4H-pyrido[2,3-f][1,2,4]triazolo[4,3-a][1,4]diazepine

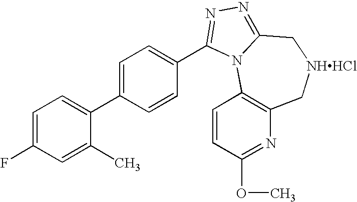

To the product of example 19 (280 mg, 0.57 mmol) and N-ethyldiisopropylamine (0.22 mL, 1.26 mmol) in dichloromethane (3 mL) at 0° C. was added 1-chloroethyl chloroformate (0.25 mL, 2.33 mmol). The resulting mixture was heated at reflux for 2 hrs. Saturated citric acid solution (3 mL) was then added and the organic layer was then separated and concentrated in vacuo. The residue was taken-up in MeOH (3 mL) and heated at reflux for 2 hrs. Sodium hydrogen carbonate solution (3 mL) was then added and the organic layer was then separated, dried over magnesium sulfate and concentrated in vacuo. The residue was purified by column chromatography eluting with neat DCM then DCM:MeOH 95: to afford the title compound as a beige solid in 11% yield, 24 mg. ¹H NMR (CD₃OD, 400 MHz), δ: 2.27 (s, 3H), 4.05 (s, 3H), 4.51 (s, 4H), 6.97 (d, 1H), 6.99-7.02 (m, 1H), 7.05-7.08 (m, 1H), 7.22-7.25 (m, 1H), 7.43 (d, 1H), 7.45-7.47 (dt, 2H), 7.65-7.67 (dt, 2H). MS APCI+ m/z 402 [MH]⁺

### EXAMPLES 28 to 34

The following compounds, of the general formula shown below, were prepared by the method that described for example 27 using the appropriate starting material.

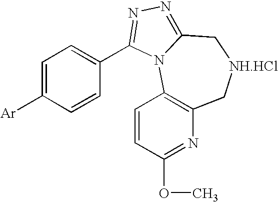

| Ex. | Ar | Data |
|---|---|---|
| 28 | 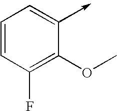 | ¹H NMR(CD₃OD, 400MHz) δ: 3.74(m, 3H), 4.06(s, 3H), 4.56(br s, 4H), 6.98(d, 1H) 7.14-7.23(m, 3H), 7.44(d, 1H), 7.70(s, 4H). MS APCl + m/z 418 [MH]⁺ |
| 29 | 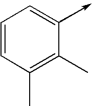 | ¹H NMR(CD₃OD, 400MHz) δ: 2.16(s, 3H), 2.34(s, 3H), 4.06(s, 3H), 4.58(m, 4H) 7.00(d, 1H), 7.04(d, 1H), 7.12-7.20(m, 2H), 7.46-7.49(m, 3H), 7.69(d, 1H) MS APCl + m/z 398 [MH]⁺ |
| 30 | 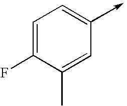 | ¹H NMR(CD₃OD, 400MHz) δ: 2.33(s, 3H), 4.06(s, 3H), 4.60(m, 4H), 6.97(d, 1H) 7.13(t, 1H), 7.49-7.53(m, 1H), 7.56-7.59(m, 1H), 7.70(d, 2H), 7.80(d, 2H) MS APCl + m/z 402 [MH]⁺ |
| 31 | 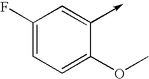 | ¹H NMR(CD₃OD, 400MHz) δ: 3.79(s, 3H), 4.05(s, 3H), 4.52(s, 4H), 6.96(d, 1H) 7.09-7.12(m, 3H), 7.43(d, 1H), 7.62-7.68(m, 4H) MS APCl + m/z 418 [MH]⁺ |
| 32 | 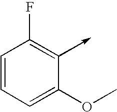 | ¹H NMR(CD₃OD, 400MHz) δ: 3.79(s, 3H), 4.05(s, 3H), 4.52(s, 4H), 6.76-6.85(m, 1H) 6.93(d, 1H), 6.98(d, 1H), 7.34-7.39(q, 1H), 7.45(d, 1H), 7.50-7.52(m, 2H), 7.63-7.65(m, 1H) MS APCl + m/z 418 [MH]⁺ |
| 33 | 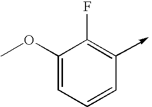 | ¹H NMR(CD₃OD, 400MHz) δ: 3.91(s, 3H), 4.05(s, 3H), 4.51(s, 4H), 6.97(d, 1H) 7.03-7.07(td, 1H), 7.12-7.22(m, 2H), 7.41-7.43(d, 1H), 7.68(s, 4H) MS APCl + m/z 418 [MH]⁺ |
| 34 | 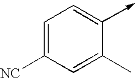 | ¹H NMR(CD₃OD, 400MHz) δ: 2.32(s, 3H), 4.05(s, 3H), 4.51(s, 4H), 6.96(d, 1H) 7.42(t, 2H), 7.50(d, 2H), 7.62-7.64(m, 1H), 7.69-7.73(m, 3H) MS APCl + m/z 461 [MH]⁺ |

### EXAMPLES 35 to 42

The following compounds, of the general formula shown below, were prepared by the method that described for example 5 using the appropriate starting material.

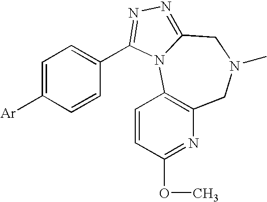

| Ex. | Ar | Data |
|---|---|---|
| 35 | 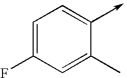 | ¹H NMR(CDCl₃, 400MHz) δ: 2.25(s, 3H), 2.62(s, 3H), 3.76(s, 2H), 3.85(s, 2H) 4.03(s, 3H), 6.71(d, 1H), 6.92-7.00(m, 2H), 7.17-7.20(m, 2H), 7.31-7.34(dt, 2H), 7.56-7.59(m, 1H). MS APCl + m/z 416 [MH]⁺ |
| 36 | 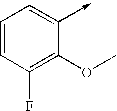 | ¹H NMR(CDCl₃, 400MHz) δ: 2.58(s, 3H), 3.72(d, 5H), 3.81(s, 2H), 4.02(s, 3H) 6.89(d, 1H), 7.05-7.13(m, 3H), 7.18(d, 1H), 7.55-7.60(m, 4H) MS APCl + m/z 432 [MH]⁺ |
| 37 | 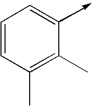 | ¹H NMR(CDCl₃, 400MHz) δ: 2.14(s, 3H), 2.34(s, 3H), 2.64(s, 3H), 3.76(br s, 2H) 3.88(br s, 2H), 4.04(s, 3H), 6.73(d, 1H), 7.07.709(dd, 1H), 7.14-7.23(m, 3H), 7.33-7.36(dt, 2H), 7.56-7.59(dt, 2H). MS APCl + m/z 412 [MH]⁺ |
| 38 | 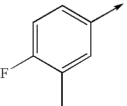 | ¹H NMR(CDCl₃, 400MHz) δ: 2.34(s, 3H), 2.61(s, 3H), 3.76(br s, 2H), 3.84(br s, 2H) 4.03(s, 3H), 6.98(d, 1H), 7.08(t, 1H), 7.16(d, 2H), 7.35-7.43(m, 2H), 7.55-7.60(m, 4H). MS APCl + m/z 416 [MH]⁺ |
| 39 | 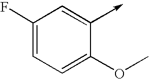 | ¹H NMR(CDCl₃, 400MHz) δ: 2.63(s, 3H), 3.78(s, 5H), 3.86(br s, 2H), 4.03(s, 3H) 6.71(d, 1H), 6.90-6.92(dd, 2H), 6.99-7.07(m, 2H), 7.21(d, 1H), 7.57(brs, 4H) MS APCl + m/z 432 [MH]⁺ |
| 40 | 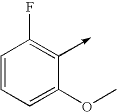 | ¹H NMR(CDCl₃, 400MHz) δ: 2.60(s, 3H), 3.74(s, 2H), 3.78(s, 3H), 3.83(s, 2H) 4.03(s, 3H), 6.71(d, 1H), 6.77-6.82(m, 2H), 6.84(d, 1H), 7.27-7.31(m, 1H), 7.46-7.47(dd, 2H), 7.56-7.60(dt, 2H). MS APCl + m/z 432 [MH]⁺ |
| 41 | 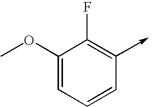 | ¹H NMR(CDCl₃, 400MHz) δ: 2.67(br s, 3H), 3.81(br s, 2H), 3.91(br s, 2H), 3.94(s, 3H) 4.04(s, 3H), 6.73(d, 1H), 6.97-7.03(m, 2H), 7.13-7.17(td, 1H), 7.21(d, 1H), 7.61(br s, 4H). MS APCl + m/z 432 [MH]⁺ |
| 42 | 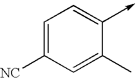 | ¹H NMR(CDCl₃, 400MHz) δ: 2.29(s, 3H), 2.64(s, 3H), 3.78(s, 2H), 3.87(s, 2H) 4.04(s, 3H), 6.73(d, 1H), 7.19(d, 1H), 7.32-7.36(m, 3H), 7.54-7.64(m, 4H) MS APCl + m/z 423 [MH]⁺ |

The following compounds of formula (I) can also be prepared using the methods disclosed above:
8-methoxy-5-methyl-1-[5-(2-methylphenyl)pyrazin-2-yl]-5,6-dihydro-4H-pyrido[2,3-f][1,2,4]triazolo[4,3-a][1,4]diazepine;
8-methoxy-1-[5-(2-methoxyphenyl)pyrazin-2-yl]-5-methyl-5,6-dihydro-4H-pyrido[2,3-f][1,2,4]triazolo[4,3-a][1,4]diazepine;
1-[5-(4-fluoro-2-methylphenyl)pyrazin-2-yl]-8-methoxy-5-methyl-5,6-dihydro-4H-pyrido[2,3-f][1,2,4]triazolo[4,3-a][1,4]diazepine;
1-[5-(3-fluoro-2-methoxyphenyl)pyrazin-2-yl]-8-methoxy-5-methyl-5,6-dihydro-4H-pyrido[2,3-f][1,2,4]triazolo[4,3-a][1,4]diazepine;
1-[5-(2,3-dimethylphenyl)pyrazin-2-yl]-8-methoxy-5-methyl-5,6-dihydro-4H-pyrido[2,3-f][1,2,4]triazolo[4,3-a][1,4]diazepine;
1-[5-(4-fluoro-3-methylphenyl)pyrazin-2-yl]-8-methoxy-5-methyl-5,6-dihydro-4H-pyrido[2,3-f][1,2,4]triazolo[4,3-a][1,4]diazepine;
1-[5-(5-fluoro-2-methoxyphenyl)pyrazin-2-yl]-8-methoxy-5-methyl-5,6-dihydro-4H-pyrido[2,3-f][1,2,4]triazolo[4,3-a][1,4]diazepine;
1-[5-(2-fluoro-6-methoxyphenyl)pyrazin-2-yl]-8-methoxy-5-methyl-5,6-dihydro-4H-pyrido[2,3-f][1,2,4]triazolo[4,3-a][1,4]diazepine;
1-[5-(2-fluoro-3-methoxyphenyl)pyrazin-2-yl]-8-methoxy-5-methyl-5,6-dihydro-4H-pyrido[2,3-f][1,2,4]triazolo[4,3-a][1,4]diazepine; and
4-[5-(8-methoxy-5-methyl-5,6-dihydro-4H-pyrido[2,3-f][1,2,4]triazolo[4,3-a][1,4]diazepin-1-yl)pyrazin-2-yl]-3-methylbenzonitrile.

1. A compound of formula (I) 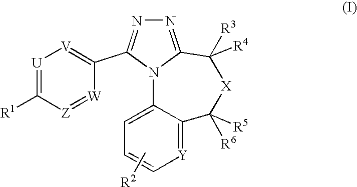
wherein
U, V, W and Z are each independently N or CR⁷;
Y is N or CH;
X is O or NR;
R¹ is selected from (i) phenyl, which is optionally substituted by one or more groups each independently selected from halo, hydroxy, CN, NO₂, (C₁-C₆)alkyl, (C₁-C₆)alkoxy, (C₁-C₆)haloalkyl, (C₁-C₆)haloalkoxy, COR⁹, CO₂R⁹, NR⁹R¹⁰ and CONR⁹R¹⁰, (ii) a five to seven membered heteroaromatic ring containing 1 to 3 heteroatoms independently selected from nitrogen, oxygen and sulfur, which is optionally substituted by one or more groups independently selected from halo, hydroxy, CN, NO₂, (C₁-C₆)alkyl, (C₁-C₆)alkoxy, (C₁-C₆)haloalkyl, (C₁-C₆)haloalkoxy, COR⁹, CO₂R⁹, NR⁹R¹⁰ and CONR⁹R¹⁰, and (iii) (C₁-C₆)alkoxy, which is optionally substituted by one or more substituents each independently selected from (C₁-C₆)alkoxy, halo, hydroxy and phenyl;
R² is hydrogen, halo, hydroxy, CN, NO₂, (C₁-C₆)alkyl, (C₁-C₆)alkoxy, (C₁-C₆)haloalkyl, (C₁-C₆)haloalkoxy, COR⁹, CO₂R⁹, NR⁹R¹⁰ or CONR⁹R¹⁰;
R³, R⁴, R⁵, and R⁶ are each independently hydrogen or (C₁-C₆)alkyl;
R⁷ is independently selected from hydrogen, halo, hydroxy, CN, NO₂, (C₁-C₆)alkyl, (C₁-C₆)alkoxy, (C₁-C₆)haloalkyl, (C₁-C₆)haloalkoxy, COR⁹, CO₂R⁹, NR⁹R¹⁰ or CONR⁹R¹⁰;
R⁸ is hydrogen or (C₁-C₆)alkyl, CO(C₁-C₆)alkyl, CO₂(C₁-C₆)alkyl or SO₂(C₁-C₆)alkyl, each of which is optionally substituted by one or more groups independently selected from halo, hydroxy, (C₁-C₆)alkoxy, CN, NO₂ and phenyl; and
R⁹ and R¹⁰ are each independently hydrogen or (C₁-C₆)alkyl;
a tautomer thereof or a pharmaceutically acceptable salt or solvate of said compound or tautomer thereof.

2. A compound according to claim 1 wherein U, V, W and Z are CH.

3. A compound according to claim 1 wherein U, V and Z are CH and W is N.

4. A compound according to any one of claims 1 to 3 wherein Y is N.

5. A compound according to any one of claims 1 to 3 wherein Y is CH.

6. A compound according to any one of claims 1 to 3 wherein R¹ is selected from
(i) phenyl, which is optionally substituted by one or more groups each independently selected from halo, (C₁-C₆)alkyl, (C₁-C₆)alkoxy, and cyano,
(ii) a six-membered heteroaromatic ring containing 1 to 2 nitrogen atoms, which is optionally substituted by one or more groups each independently selected from halo, (C₁-C₆)alkyl, (C₁-C₆)alkoxy, and cyano, and
(iii) (C₁-C₆)alkoxy, which is optionally substituted by one or more substituents each independently selected from (C₁-C₆)alkoxy, halo, hydroxy and phenyl.

7. A compound according to claim 6 wherein R¹ is selected from phenyl and pyridyl, each of which is optionally substituted by one or more groups each independently selected from halo, (C₁-C₆)alkyl, (C₁-C₆)alkoxy, and cyano.

8. A compound according to claim 7 wherein R¹ is selected from phenyl which is optionally substituted by one or two groups each independently selected from fluoro, methyl, methoxy and cyano, and pyridyl, which is substituted by methyl.

9. A compound according to any one of claims 1 to 3 wherein R² is methoxy, which is situated on the carbon adjacent to group Y.

10. A compound according to any one of claims 1 to 3 wherein R³, R⁴, R⁵ and R⁶ are hydrogen.

11. A compound according to any one of claims 1 to 3 wherein R⁸ is hydrogen, CH₃, COCH₃, CO₂CH₃, SO₂CH₃ or benzyl.

12. A compound according to claim 1 which is selected from
8-Methoxy-1-(2′-methoxybiphenyl-4-yl)-4H,6H-[1,2,4]triazolo[4,3-a][4,1 ]benzoxazepine,
8-Methoxy-1-(2′-methoxybiphenyl-4-yl)-5,6-dihydro-4H-pyrido[2,3-f][1,2,4]triazolo[4,3-a][1,4]diazepine,
8-Methoxy-5-methyl-1-(2′-methoxybiphenyl-4-yl)-5,6-dihydro-4H-pyrido[2,3-f][1,2,4]triazolo[4,3-a][1,4]diazepine,
5-Acetyl-8-methoxy-1-(2′-methoxybiphenyl-4-yl)-5,6-dihydro-4H-pyrido[2,3-f][1,2,4]triazolo[4,3-a][1,4]diazepine,
8-Methoxy-1-(2′-methylbiphenyl-4-yl)-4H,6H-[1,2,4]triazolo[4,3-a][4,1]benzoxazepine,
8-Methoxy-1-(2′-methylbiphenyl-4-yl)-5,6-dihydro-4H-pyrido[2,3-f][1,2,4]triazolo[4,3-a][1,4]diazepine,
8-Methoxy-5-methyl-1-(2-methylbiphenyl-4-yl)-5,6-dihydro-4H-pyrido[2,3-f][1,2,4]triazolo[4,3-a][1,4]diazepine, and
5-Acetyl-8-methoxy-1-(2′-methylbiphenyl-4-yl)-5,6-dihydro-4H-pyrido[2,3-f][1,2,4]triazolo[4,3-a][1,4]diazepine,
and tautomers thereof and pharmaceutically acceptable salts or solvate of said compound or tautomer.

13. A pharmaceutical composition comprising a compound of formula (I) as claimed in any one of claims 1 to 3, or pharmaceutically acceptable salts or solvate thereof, and a pharmaceutically acceptable diluent or carrier.

14. (canceled)

15. A method of treatment of a disorder or condition where inhibition of oxytocin is known, or can be shown, to produce a beneficial effect, in a mammal, comprising administering to said mammal a therapeutically effective amount of a compound of formula (I) as claimed in any one of claims 1 to 3, or a pharmaceutically acceptable salt or thereof.

16. (canceled)

17. A method according to claim 15 wherein the disorder or condition is selected from sexual dysfunction, male sexual dysfunction, female sexual dysfunction, hypoactive sexual desire disorder, sexual arousal disorder, orgasmic disorder, sexual pain disorder, premature ejaculation, preterm labour, complications in labour, appetite and feeding disorders, benign prostatic hyperplasia, premature birth, dysmenorrhoea, congestive heart failure, arterial hypertension, liver cirrhosis, nephrotic hypertension, occular hypertension, obsessive compulsive disorder and neuropsychiatric disorders.

18. A method or use according to claim 17 wherein the disorder or condition is selected from sexual arousal disorder, orgasmic disorder, sexual pain disorder and premature ejaculation.
